(19) United States
(12) Patent Application Publication
(10) Pub. No.: US 2022/0290169 A1
(43) Pub. Date: Sep. 15, 2022

(54) FLOWERING TIME GENES AND METHODS OF USE

(71) Applicants: PIONEER OVERSEAS CORPORATION, JOHNSTON, IA (US); SINOBIOWAY BIO-AGRICULTURE GROUP CO. LTD., BEIJING (CN)

(72) Inventors: GUIHUA LU, SAN DIEGO, CA (US); GUANGWU CHEN, BEIJING (CN); RONGRONG JIAO, BEIJING (CN); JUNHUA LIU, BEIJING (CN); GUANFAN MAO, BEIJING (CN); CHAO SONG, BEIJING (CN); CHANGGUI WANG, BEIJING (CN); GUOKUI WANG, BEIJING (CN); WEI WANG, BEIJING (CN); XIPING WANG, BEIJING (CN); YUZHEN ZHENG, ZHENGZHOU CITY (CN); ZHANCHUN ZHOU, BEIJING (CN)

(73) Assignees: PIONEER OVERSEAS CORPORATION, JOHNSTON, IA (US); SINOBIOWAY BIO-AGRICULTURE GROUP CO. LTD., BEIJING (CN)

(21) Appl. No.: 17/632,374

(22) PCT Filed: Aug. 19, 2019
(86) PCT No.: PCT/CN2019/101286

§ 371 Date: Feb. 2, 2022

(51) Int. Cl. C12N 15/82; C07K 14/415

(52) U.S. Cl. CPC C12N 15/827; C07K 14/415; C12N 15/8218

(57) ABSTRACT

Isolated polynucleotides and polypeptides, and recombinant DNA constructs are useful for conferring delayed or accelerated flowering time and maturity. Compositions (such as plants or seeds) comprise these recombinant DNA constructs; and methods utilize these recombinant DNA constructs. The recombinant DNA constructs comprise a polynucleotide operably linked to a promoter that is functional in a plant, wherein said polynucleotides encode late flowering polypeptides.

### FIELD

This disclosure relates to the field of plant breeding and genetics and relates to recombinant DNA constructs useful for regulating flowering time and/or heading date of plants, and methods for the control of flowering time, heading date and/or maturity in plants.

### BACKGROUND

The growth phase of plants generally includes a vegetative growth phase and a reproductive growth phase. The transition from vegetative to reproductive growth is affected by various flowering signals. The flowering signals are affected by various factors, such as genetic factors (e.g., genotype) and environmental factors (e.g., photoperiod and light intensity) (Dung et al., Theoretical and Applied Genetics, 97: 714-720 (1998)).

Flowering time or heading date is an important agronomic trait and is a critical determinant of the distribution and regional adaptability of plants. Accelerating or delaying the onset of flowering can be useful to farmers and seed producers.

Accordingly, there is a need to develop new compositions and methods for altering the flowering characteristics of the target plant (e.g., cereals, rice and maize, in warmer climatic zones, and wheat, barley, oats and rye in more temperature climates). This disclosure provides such compositions and methods.

### SUMMARY

The following embodiments are among those encompassed by the disclosure:

In one embodiment, the present disclosure includes an isolated polynucleotide, encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117,119, 121, 123, 125, or 127, wherein increased expression of the polynucleotide in a plant delays flowering time. In certain embodiments, the isolated polynucleotide encodes an amino acid sequence of SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105,107, 109, 111, 113, 115,117, 119, 121, 123, 125, or 127. In certain embodiments, the isolated polynucleotide comprises the nucleotide sequence of SEQ ID NO: 1, 2, 4, 5, 7, 8, 10, 11, 13, 14, 16, 17, 19, 20, 22, 23, 25, 26, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, or 126. In certain embodiments, increased expression of the polynucleotide in a plant delays the maturity of the plant.

The present disclosure also provides a recombinant DNA construct comprising an isolated polynucleotide operably linked to at least one heterologous regulatory element, wherein the polynucleotide encodes a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

The present disclosure further provides a modified plant or seed having increased expression or activity of at least one polynucleotide encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127. In certain embodiments, the modified plant or seed comprises in its genome a recombinant DNA construct comprising a polynucleotide operably linked to at least one heterologous regulatory element, wherein the polynucleotide encodes a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127. In certain embodiments, the modified plant exhibits delayed flowering time and/or maturity when grown under field conditions compared to a control plant.

In certain embodiments, the modified plant or seed comprises a targeted genetic modification at a genomic locus comprising a polynucleotide encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, wherein the targeted genetic modification increases the expression and/or activity of the polypeptide. In certain embodiments, the modified plant exhibits delayed flowering time and late maturity when grown under field conditions compared to a control plant.

The present disclosure further provides a modified plant or seed having decreased expression or activity of at least one polynucleotide encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127. In certain embodiments, the modified plant or seed comprises in its genome an RNAi construct that targets a polynucleotide encoding a polypeptide having an amino acid sequence of at least 80% sequence identity sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127. In certain embodiments, the modified plant exhibits early flowering time and/or maturity when grown under field conditions compared to a control plant.

In certain embodiments, the modified plant or seed comprises a targeted genetic modification at a genomic locus comprising a polynucleotide encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, wherein the targeted genetic modification decreases the expression and/or activity of the polypeptide. In certain embodiments, the modified plant exhibits early flowering time and/or early maturity when grown under field conditions compared to a control plant.

In certain embodiments, the plant for use in the compositions and methods provided herein is selected from the group consisting of rice, maize, soybean, sunflower, sorghum, canola, wheat, alfalfa, cotton, barley, millet, sugar cane and switchgrass.

Also provided are methods for delaying flowering time in a plant, the method comprising increasing the expression of at least one polynucleotide encoding a polypeptide with amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127 in the plant, wherein the plant exhibits late flowering time when compared to the control plant.

In certain embodiments, the method for delaying flowering time comprises: (a) introducing into a regenerable plant cell a recombinant DNA construct comprising a polynucleotide operably linked to at least one heterologous regulatory element, wherein the polynucleotide encodes a polypeptide having an amino acid sequence of at least 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and (b) generating the plant, wherein the plant comprises in its genome the recombinant DNA construct.

In certain embodiments, the method for delaying flowering time comprises: (a) introducing into a regenerable plant cell a targeted genetic modification at a genomic locus comprising a polynucleotide encoding a polypeptide having an amino acid sequence of at least 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and (b) generating the plant, wherein the plant comprises in its genome the introduced genetic modification and has increased expression and/or activity of the polypeptide. In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

Also provided are methods for accelerating flowering time in a plant, the method comprising decreasing the expression of at least one polynucleotide encoding a polypeptide with amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127 in the plant, wherein the plant exhibits early flowering time when compared to the control plant.

In certain embodiments, the method for accelerating flowering time or early maturity comprises: (a) introducing into a regenerable plant cell a RNAi construct of comprising a hairpin structure polynucleotide encoding a polypeptide having an amino acid sequence of at least 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and (b) generating the plant, wherein the plant comprises in its genome the introduced genetic modification and has decreased expression and/or activity of the polypeptide.

In certain embodiments, the method for accelerating flowering time comprises: (a) introducing into a regenerable plant cell a targeted genetic modification at a genomic locus comprising a polynucleotide encoding a polypeptide having an amino acid sequence of at least 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and (b) generating the plant, wherein the plant comprises in its genome the introduced genetic modification and has decreased expression and/or activity of the polypeptide.

In certain embodiments, the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), an engineered site-specific meganucleases, or an Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at 80% sequence identity, when compared to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

### BRIEF DESCRIPTION OF THE DRAWINGS AND SEQUENCE LISTING

The disclosure can be more fully understood from the following detailed description and the accompanying Sequence Listing which form a part of this application. The sequence descriptions and sequence listing attached here to comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

TABLE 1
Sequence Listing Descriptions

| Source/Plant | Clone | SEQ ID NO: | SEQ ID NO: |
|---|---|---|---|
| species | Designation | (Nucleotide) | (Amino Acid) |
| Oryza sativa | OsHIS | 1, 2 | 3 |
| Oryza sativa | OsDN-FTG1 | 4, 5 | 6 |
| Oryza sativa | OsWRKY76 | 7, 8 | 9 |
| Oryza sativa | OSMYB77 | 10, 11 | 12 |
| Oryza sativa | OsDN-FTG2 | 13, 14 | 15 |
| Oryza sativa | OsENA1 | 16, 17 | 18 |
| Oryza sativa | OsGRF1 | 19, 20 | 21 |
| Oryza sativa | OsHIP14 | 22, 23 | 24 |
| Oryza sativa | OsDN-FTG3 | 25, 26 | 27 |
| Artificial | Gene Clone Primers | 28-45 | n/a |
| Artificial | RT-PCR Primers | 46-59 | n/a |
| Oryza sativa | OsHIS Paralog | 60 | 61 |
| Zea mays | OsHIS Homolog | 62 | 63 |
| Sorghum bicolor | OsHIS Homolog | 64 | 65 |
| Arabidopsis thaliana | OsHIS Homolog | 66 | 67 |
| Glycine max | OsHIS Homolog | 68 | 69 |
| Zea mays | OsDN-FTG1 Homolog | 70 | 71 |
| Sorghum bicolor | OsDN-FTG1 Homolog | 72 | 73 |
| Arabidopsis thaliana | OsDN-FTG1 Homolog | 74 | 75 |
| Oryza sativa | OsWRKY76 Paralog | 76 | 77 |
| Zea mays | OsWRKY76 Homolog | 78 | 79 |
| Sorghum bicolor | OsWRKY76 Homolog | 80 | 81 |
| Arabidopsis thaliana | OsWRKY76 Homolog | 82 | 83 |
| Glycine max | OsWRKY76 Homolog | 84 | 85 |
| Oryza sativa | OsMYB77 Paralog | 86 | 87 |
| Zea mays | OsMYB77 Homolog | 88 | 89 |
| Sorghum bicolor | OsMYB77 Homolog | 90 | 91 |
| Arabidopsis thaliana | OsMYB77 Homolog | 92 | 93 |
| Glycine max | OsMYB77 Homolog | 94 | 95 |
| Oryza sativa | OsDN-FTG2 Paralog | 96 | 97 |
| Oryza sativa | OsENA1 Paralog | 98 | 99 |
| Zea mays | OsENA1 Homolog | 100 | 101 |
| Sorghum bicolor | OsENA1 Homolog | 102 | 103 |
| Glycine max | OsENA1 Homolog | 104 | 105 |
| Oryza sativa | OsGRF1 Paralog | 106 | 107 |
| Zea mays | OsGRF1 Homolog | 108 | 109 |
| Sorghum bicolor | OsGRF1 Homolog | 110 | 111 |
| Arabidopsis thaliana | OsGRF1 Homolog | 112 | 113 |
| Glycine max | OsGRF1 Homolog | 114 | 115 |
| Oryza sativa | OsHIP14 Paralog | 116 | 117 |
| Zea mays | OsHIP14 Homolog | 118 | 119 |
| Sorghum bicolor | OsHIP14 Homolog | 120 | 121 |
| Arabidopsis thaliana | OsHIP14 Homolog | 122 | 123 |
| Glycine max | OsHIP14 Homolog | 124 | 125 |
| Oryza sativa | OsDN-FTG3 Paralog | 126 | 127 |

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

### Definitions

“Flowering time” also referred to herein as “first heading time” is defined as the days from sowing the seed to the first heading date and/or 50% heading date of the plant. The first heading date is the date when the first panicle, usually the main stem panicle, heads out the sheath of flag leaf. The 50% heading date is the date when 50% young panicles head out the sheath of flag leaf for plants in one row of the same line.

“Late flowering or delayed flowering time” of a plant refers to any measurable delay in flowering time relative to a reference or a control plant when grown under same conditions.

“Early flowering or accelerated flowering time” of a plant refers to any measurable decrease in flowering time relative to a reference or control plant when grown under same conditions.

“Maturity” is the date when 90% glume, grain spikelet axis or vice glume become yellow from appearance, which is the best harvest period.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, heading date, maturity date, panicle size, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control”, “control plant” or “control plant cell” or the like provides a reference point for measuring changes in phenotype of a subject plant or plant cell in which genetic alteration, such as transformation, has been affected as to a gene of interest. For example, a control plant may be a plant having the same genetic background as the subject plant except for the genetic alteration that resulted in the subject plant or cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Modified plant” includes reference to a plant which comprises within its genome a heterologous polynucleotide or modified gene or promoter. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Recombinant DNA construct” refers to a combination of nucleic acid fragments that are not normally found together in nature. Accordingly, a recombinant DNA construct may comprise regulatory elements and coding sequences that are derived from different sources, or regulatory elements and coding sequences derived from the same source, but arranged in a manner different than that normally found in nature.

“Regulatory elements” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory elements may include, but are not limited to, promoters, translation leader sequences, introns, and poly-adenylation recognition sequences. The terms “regulatory sequence” and “regulatory element” and “regulatory region” are used interchangeably herein.

“Promoter” refers to a nucleic acid fragment capable of controlling transcription of another nucleic acid fragment. “Promoter functional in a plant” is a promoter capable of controlling transcription of genes in plant cells whether its origin is from a plant cell or not. “Tissue-specific promoter” and “tissue-preferred promoter” refers to a promoter that is expressed predominantly but not necessarily exclusively in one tissue or organ, but that may also be expressed in one specific cell or cell type. “Developmentally regulated promoter” is a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

RNA interference (RNAi) refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

RNAi constructs comprise nucleic acids that target and decrease expression of a gene of interest, and include, without limitation, co-suppression constructs, antisense constructs, viral-suppression constructs, hairpin suppression constructs, stem-loop suppression constructs, double-stranded RNA-producing constructs, siRNA constructs, and miRNA constructs.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

As used herein “increased”, “increase”, or the like refers to any detectable increase in an experimental group (e.g., plant with a DNA modification described herein) as compared to a control group (e.g., wild-type plant that does not comprise the DNA modification). Accordingly, increased expression of a protein comprises any detectable increase in the total level of the protein in a sample and can be determined using routine methods in the art such as, for example, Western blotting and ELISA.

As used herein “decreased”, “decrease”, or the like refers to any detectable decrease in an experimental group (e.g., plant with a DNA modification described herein) as compared to a control group (e.g., wild-type plant that does not comprise the DNA modification). Accordingly, decreased expression of a protein comprises any detectable decrease in the total level of the protein in a sample and can be determined using routine methods in the art such as, for example, Western blotting and ELISA.

As used herein, “yield” refers to the amount of agricultural production harvested per unit of land, and may include reference to bushels per acre or kilograms per mu of a crop at harvest, as adjusted for grain moisture (e.g., typically 15% for maize, 13.5% for rice). Grain moisture is measured in the grain at harvest. The adjusted test weight of grain is determined to be the weight in pounds per bushel or grams per plant, adjusted for grain moisture level at harvest.

As used herein, “sequence identity” or “identity” in the context of two polynucleotides or polypeptide sequences refer to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” is calculated by determining the number of positions at which the identical nucleic acid base or amino acid residue occurs in both sequences to yield the number of matched positions, dividing the number of matched positions by the total number of positions in the window of comparison, and multiplying the result by 100.

Unless stated otherwise, multiple alignments of the sequences provided herein are performed using the Clustal V method of alignment (Higgins and Sharp. (1989) CABIOS. 5:151-153) with the default parameters (GAP PENALTY=10, GAP LENGTH PENALTY=10). Default parameters for pairwise alignments and calculation of percent identity of amino acid sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

### Compositions:

### A. Polynucleotides and Polypeptides

The present disclosure provides polynucleotides encoding the following polypeptides: HIS (core histone H2A/H2B/H3/H4, putative, expressed); DN-FTG1 (expressed protein); WRKY76 (WRKY76, expressed); MYB77 (MYB transcription factor TaMYB1, putative, expressed); DN-FTG2 (expressed protein); ENA1 (exonuclease, putative, expressed); GRF1 (growth-regulating factor, putative, expressed); HIP14 (zinc finger, C3HC4 type domain containing protein, expressed); and DN-FTG3 (expressed protein).

One aspect of the disclosure provides a polynucleotide encoding a polypeptide comprising an amino acid sequence that is at least 80% identical (e.g. 81%, 82%, 83%, 84%, 85%, 86%, 87%, 88%, 89%, 90%, 91%, 92%, 93%, 94%, 95%, 96%, 97%, 98%, 99%, or 100%) to the amino acid sequence of any one of SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115,117, 119, 121, 123, 125, or 127.

“OsHIS” refers to a rice polypeptide that confers late flowering phenotype when overexpressed. The OsHIS polypeptides (SEQ ID NO: 3) are encoded by the coding sequences (CDS) (SEQ ID NO: 2) or nucleotide sequence (SEQ ID NO: 1) at rice gene locus LOC_Os03g14669.2, which is annotated as “core histone H2A/H2B/H3/H4, putative, expressed” in TIGR. “HIS polypeptide” refers herein to the OsHIS polypeptide and its paralogs (e.g., SEQ ID NO: 61 encoded by SEQ ID NO: 60) or its homologs from other organisms, such as maize (SEQ ID NO: 63 encoded by SEQ ID NO: 62), sorghum (SEQ ID NO: 65 encoded by SEQ ID NO: 64), Arabidopsis (SEQ ID NO: 67 encoded by SEQ ID NO: 66), and soybean (SEQ ID NO: 69 encoded by SEQ ID NO: 68).

“OsDN-FTG1” refers to a rice polypeptide that confers late flowering phenotype when overexpressed. The OsDN-FTG1 polypeptide (SEQ ID NO: 6) is encoded by the coding sequence (CDS) (SEQ ID NO: 5) or nucleotide sequence (SEQ ID NO: 4) at rice gene locus LOC_Os01g04010.1, which is annotated as “expressed protein” in TIGR. “DN-FTG1 polypeptide” refers herein to the OsDN-FTG1 polypeptide and its paralogs or homologs from other organisms, such as maize (SEQ ID NO: 71 encoded by SEQ ID NO: 70), sorghum (SEQ ID NO: 73 encoded by SEQ ID NO: 72), and Arabidopsis (SEQ ID NO: 75 encoded by SEQ ID NO: 74).

“OsWRKY76” refers to a rice polypeptide that confers late flowering phenotype when overexpressed. The OsWRKY76 polypeptide (SEQ ID NO: 9) is encoded by the coding sequence (CDS) (SEQ ID NO: 8) or nucleotide sequence (SEQ ID NO: 7) at rice gene locus LOC_Os09g25060.1, which is annotated as “WRKY76, expressed” in TIGR. “WRKY76 polypeptide” refers herein to the OsWRKY76 polypeptide and its paralogs (SEQ ID NO: 77 encoded by SEQ ID NO: 76) or homologs from other organisms, such as maize (SEQ ID NO: 79 encoded by SEQ ID NO: 78); sorghum (SEQ ID NO: 81 encoded by SEQ ID NO: 80); Arabidopsis (SEQ ID NO: 83 encoded by SEQ ID NO: 82); and soybean (SEQ ID NO: 85 encoded by SEQ ID NO: 84).

“OsMYB77” refers to a rice polypeptide that confers late flowering phenotype when overexpressed. The OsMYB77 polypeptide (SEQ ID NO: 12) is encoded by the coding sequence (CDS) (SEQ ID NO: 11) or nucleotide sequence (SEQ ID NO: 10) at rice gene locus LOC_Os06g43090.1, which is annotated as “MYB transcription factor TaMYB1, putative, expressed” in TIGR. “MYB77 polypeptide” refers herein to the OsMYB77 polypeptide and its paralogs (e.g., SEQ ID NO: 87 encoded by SEQ ID NO: 86) or homologs from other organisms, such as maize (SEQ ID NO: 89 encoded by SEQ ID NO: 88), sorghum (SEQ ID NO: 91 encoded by SEQ ID NO: 90), Arabidopsis (SEQ ID NO: 93 encoded by SEQ ID NO: 92), and soybean (SEQ ID NO: 95 encoded by SEQ ID NO: 94).

“OsDN-FTG2” refers to a rice polypeptide that confers late flowering phenotype when overexpressed. The OsDN-FTG2 polypeptide (SEQ ID NO: 15) is encoded by the coding sequence (CDS) (SEQ ID NO: 14) or nucleotide sequence (SEQ ID NO: 13) at rice gene locus LOC_Os03g30680.1, which is annotated as “expressed protein” in TIGR. “DN-FTG2 polypeptide” refers herein to the OsDN-FTG2 polypeptide and its paralogs (e.g., SEQ ID NO: 97 encoded by SEQ ID NO: 96) or homologs from other organisms.

“OsENA1” refers to a rice polypeptide that confers late flowering phenotype when overexpressed. The OsENA1 polypeptide (SEQ ID NO: 18) is encoded by the coding sequence (CDS) (SEQ ID NO: 17) or nucleotide sequence (SEQ ID NO: 16) at rice gene locus LOC_Os01g43080.1, which is annotated as “exonuclease, putative, expressed” in TIGR. “ENA1 polypeptide” refers herein to the OsENA1 polypeptide and its paralogs (e.g., SEQ ID NO: 99 encoded by SEQ ID NO: 98) or homologs from other organisms, such as maize (SEQ ID NO: 101 encoded by SEQ ID NO: 100), sorghum (SEQ ID NO: 103 encoded by SEQ ID NO: 102), and soybean (SEQ ID NO: 105 encoded by SEQ ID NO: 104).

“OsGRF1” refers to a rice polypeptide that confers late flowering phenotype when overexpressed. The OsGRF1 polypeptide (SEQ ID NO: 21) is encoded by the coding sequence (CDS) (SEQ ID NO: 20) or nucleotide sequence (SEQ ID NO: 19) at rice gene locus LOC_Os04g51190.1, which is annotated as “growth-regulating factor, putative, expressed” in TIGR. “GRF1 polypeptide” refers herein to the OsGRF1 polypeptide and its paralogs (e.g., SEQ ID NO: 107 encoded by SEQ ID NO: 106) or homologs from other organisms, such as maize (SEQ ID NO: 109 encoded by SEQ ID NO: 108), sorghum (SEQ ID NO: 111 encoded by SEQ ID NO: 110),), Arabidopsis (SEQ ID NO: 113 encoded by SEQ ID NO: 112), and soybean (SEQ ID NO: 115 encoded by SEQ ID NO: 114).

“OsHIP14” refers to a rice polypeptide that confers late flowering phenotype when overexpressed. The OsHIP14 polypeptide (SEQ ID NO: 24) is encoded by the coding sequence (CDS) (SEQ ID NO: 23) or nucleotide sequence (SEQ ID NO: 22) at rice gene locus LOC_Os04g55510.1, which is annotated as “zinc finger, C3HC4 type domain containing protein, expressed” in TIGR. “HIP14 polypeptide” refers herein to the OsHIP14 polypeptide and its paralogs (e.g., SEQ ID NO: 117 encoded by SEQ ID NO: 116) or homologs from other organisms, such as maize (SEQ ID NO: 119 encoded by SEQ ID NO: 118), sorghum (SEQ ID NO: 121 encoded by SEQ ID NO: 120), Arabidopsis (SEQ ID NO: 123 encoded by SEQ ID NO: 120), and soybean (SEQ ID NO: 125 encoded by SEQ ID NO: 124).

“OsDN-FTG3” refers to a rice polypeptide that confers late flowering phenotype when overexpressed. The OsDN-FTG3 polypeptide (SEQ ID NO: 27) is encoded by the coding sequence (CDS) (SEQ ID NO: 26) or nucleotide sequence (SEQ ID NO: 25) at rice gene locus LOC_Os03g61070.1, which is annotated as “expressed protein” in TIGR. “DN-FTG3 polypeptide” refers herein to the OsDN-FTG3 polypeptide and its paralogs (e.g., SEQ ID NO: 127 encoded by SEQ ID NO: 126) or homologs from other organisms.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

### B. Recombinant DNA Constructs

Also provided are recombinant DNA constructs comprising any of the polynucleotides described herein. In certain embodiments, the recombinant DNA construct further comprises at least one regulatory element. In certain embodiments the at least one regulatory element is a heterologous regulatory element. In certain embodiments, the at least one regulatory element of the recombinant DNA construct comprises a promoter. In certain embodiments, the promoter is a heterologous promoter.

A number of promoters can be used in recombinant DNA constructs of the present disclosure. The promoters can be selected based on the desired outcome, and may include constitutive, tissue-specific, inducible, or other promoters for expression in the host organism.

A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotor et al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7: L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Also contemplated are synthetic promoters which include a combination of one or more heterologous regulatory elements.

The promoter of the recombinant DNA constructs of the invention can be any type or class of promoter known in the art, such that any one of a number of promoters can be used to express the various polynucleotide sequences disclosed herein, including the native promoter of the polynucleotide sequence of interest. The promoters for use in the recombinant DNA constructs of the invention can be selected based on the desired outcome.

The recombinant DNA constructs of the present disclosure may also include other regulatory elements, including but not limited to, translation leader sequences, introns, and polyadenylation recognition sequences. In certain embodiments, a recombinant DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

### C. Plants and Plant Cells

Provided are plants, plant cells, plant parts, seed and grain comprising in its genome any of the recombinant DNA constructs described herein, so that the plants, plant cells, plant parts, seed, and/or grain have increased expression of the encoded polypeptide. In certain embodiments the plant exhibits delayed flowering time when compared to a control plant. In certain embodiments, the plant exhibits an alteration of at least one agronomic characteristic when compared to the control plant.

Also provided are plants, plant cells, plant parts, seeds, and grain comprising an introduced genetic modification at a genomic locus that encodes a polypeptide comprising an amino acid sequence that is at least 80% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127. In certain embodiments, the genetic modification increases the activity of the encoded polypeptide. In certain embodiments, the genetic modification increases the level of the encoded polypeptide. In certain embodiments, the genetic modification increases both the level and activity of the encoded polypeptide. In certain embodiments the plant exhibits delayed flowering time when compared to a control plant. In certain embodiments, the plant exhibits an alteration of at least one agronomic characteristic when compared to the control plant.

Further provided are plants, plant cells, plant parts, seed and grain comprising in its genome an RNAi construct that targets a polynucleotide encoding a polypeptide comprising an amino acid sequence that is at least 80% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, wherein the RNAi construct decreases the expression of the encoded polypeptide. In certain embodiments the plant exhibits accelerated flowering time when compared to a control plant. In certain embodiments, the plant exhibits an alteration of at least one agronomic characteristic when compared to the control plant.

Also provided are plants, plant cells, plant parts, seeds, and grain comprising an introduced genetic modification at a genomic locus that encodes a polypeptide comprising an amino acid sequence that is at least 80% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, wherein the genetic modification decreases the level and/or activity of the encoded polypeptide. In certain embodiments, the genetic modification decreases the activity of the encoded polypeptide. In certain embodiments, the genetic modification decreases the level of the encoded polypeptide. In certain embodiments, the genetic modification decreases both the level and activity of the encoded polypeptide. In certain embodiments the plant exhibits accelerated flowering time when compared to a control plant. In certain embodiments, the plant exhibits an alteration of at least one agronomic characteristic when compared to the control plant.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

D. Stacking with Other Traits of Interest

In some embodiments, the inventive polynucleotides disclosed herein are engineered into a molecular stack. Thus, the various host cells, plants, plant cells, plant parts, seeds, and/or grain disclosed herein can further comprise one or more traits of interest. In certain embodiments, the host cell, plant, plant part, plant cell, seed, and/or grain is stacked with any combination of polynucleotide sequences of interest in order to create plants with a desired combination of traits. As used herein, the term “stacked” refers to having multiple traits present in the same plant or organism of interest. For example, “stacked traits” may comprise a molecular stack where the sequences are physically adjacent to each other. A trait, as used herein, refers to the phenotype derived from a particular sequence or groups of sequences. In one embodiment, the molecular stack comprises at least one polynucleotide that confers tolerance to glyphosate. Polynucleotides that confer glyphosate tolerance are known in the art.

In certain embodiments, the molecular stack comprises at least one polynucleotide that confers tolerance to glyphosate and at least one additional polynucleotide that confers tolerance to a second herbicide.

In certain embodiments, the plant, plant cell, seed, and/or grain having an inventive polynucleotide sequence may be stacked with, for example, one or more sequences that confer tolerance to: an ALS inhibitor; an HPPD inhibitor; 2,4-D; other phenoxy auxin herbicides; aryloxyphenoxypropionate herbicides; dicamba; glufosinate herbicides; herbicides which target the protox enzyme (also referred to as “protox inhibitors”).

The plant, plant cell, plant part, seed, and/or grain having an inventive polynucleotide sequence can also be combined with at least one other trait to produce plants that further comprise a variety of desired trait combinations. For instance, the plant, plant cell, plant part, seed, and/or grain having an inventive polynucleotide sequence may be stacked with polynucleotides encoding polypeptides having pesticidal and/or insecticidal activity, or a plant, plant cell, plant part, seed, and/or grain having an inventive polynucleotide sequence may be combined with a plant disease resistance gene.

These stacked combinations can be created by any method including, but not limited to, breeding plants by any conventional methodology, or genetic transformation. If the sequences are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

### Methods:

Provided is a method for delaying flowering time and/or late maturity, in a plant, comprising increasing the expression of at least one polynucleotide encoding a polypeptide with an amino acid sequence of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

In certain embodiments, the method comprises: (a) expressing in a regenerable plant cell a recombinant DNA construct comprising a regulatory element operably linked to the polynucleotide encoding the polypeptide; and (b) generating the plant, wherein the plant comprises in its genome the recombinant DNA construct. In certain embodiments the regulatory element is a heterologous promoter.

In certain embodiments, the method comprises: (a) introducing in a regenerable plant cell a targeted genetic modification at a genomic locus that encodes the polypeptide; and (b) generating the plant, wherein the level and/or activity of the encoded polypeptide is increased in the plant. In certain embodiments the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 80% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

In certain embodiments the DNA modification is an insertion of one or more nucleotides, preferably contiguous, in the genomic locus. For example, the insertion of an expression modulating element (EME), such as an EME described in PCT/US2018/025446, in operable linkage with the gene. In certain embodiments, the targeted DNA modification may be the replacement of the endogenous polypeptide promoter with another promoter known in the art to have higher expression. In certain embodiments, the targeted DNA modification may be the insertion of a promoter known in the art to have higher expression into the 5′UTR so that expression of the endogenous polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

Provided is a method for accelerating flowering time and/or early maturity, in a plant, comprising decreasing the expression of at least one polynucleotide encoding a polypeptide with an amino acid sequence of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

In certain embodiments, the method comprises: (a) expressing in a regenerable plant cell an RNAi construct that decreases the expression of a polynucleotide encoding a polypeptide having an amino acid sequence of at least 80% sequence identity sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and (b) generating the plant, wherein expression of the polypeptide is decreased compared to a control plant.

In certain embodiments, the method comprises: (a) introducing in a regenerable plant cell a targeted genetic modification at a genomic locus that encodes the polypeptide; and (b) generating the plant, wherein the level and/or activity of the encoded polypeptide is decreased in the plant. In certain embodiments the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute. In certain embodiments, the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 80% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

The plant for use in the inventive methods can be any plant species described herein. In certain embodiments, the plant is maize, soybean, or rice.

Various methods can be used to introduce a sequence of interest into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome.

Transformed plant cells which are derived by plant transformation techniques, including those discussed above, can be cultured to regenerate a whole plant which possesses the transformed genotype (i.e., an inventive polynucleotide), and thus the desired phenotype, such as increased yield. For transformation and regeneration of maize see, Gordon-Kamm et al., The Plant Cell, 2:603-618 (1990).

Various methods can be used to introduce a genetic modification at a genomic locus that encodes a polypeptide disclosed herein into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In some embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

In addition to modification by a double strand break technology, modification of one or more bases without such double strand break are achieved using base editing technology, see e.g., Gaudelli et al., (2017) Programmable base editing of A*T to G*C in genomic DNA without DNA cleavage. Nature 551(7681):464-471; Komor et al., (2016) Programmable editing of a target base in genomic DNA without double-stranded DNA cleavage, Nature 533(7603):420-4.

These fusions contain dCas9 or Cas9 nickase and a suitable deaminase, and they can convert e.g., cytosine to uracil without inducing double-strand break of the target DNA. Uracil is then converted to thymine through DNA replication or repair. Improved base editors that have targeting flexibility and specificity are used to edit endogenous locus to create target variations and improve grain yield. Similarly, adenine base editors enable adenine to inosine change, which is then converted to guanine through repair or replication. Thus, targeted base changes i.e., C·G to T·A conversion and A·T to G·C conversion at one more location made using appropriate site-specific base editors.

In an embodiment, base editing is a genome editing method that enables direct conversion of one base pair to another at a target genomic locus without requiring double-stranded DNA breaks (DSBs), homology-directed repair (HDR) processes, or external donor DNA templates. In an embodiment, base editors include (i) a catalytically impaired CRISPR-Cas9 mutant that are mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; and (iv) nickase activity to cleave the non-edited DNA strand, followed by cellular DNA repair processes to replace the G-containing DNA strand.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

### EXAMPLES

The following are examples of specific embodiments of some aspects of the invention. The examples are offered for illustrative purposes only and are not intended to limit the scope of the invention in any way.

### Example 1

### Cloning and Vector Construction of Late Flowering Genes

A binary construct that contains four multimerized enhancers elements derived from the Cauliflower Mosaic Virus 35S (CaMV 35S) promoter was used, and the rice activation tagging population was developed from four Japonica (Oryza sativa ssp. Japonica) varieties (Zhonghua 11, Chaoyou 1, Taizhong 65 and Nipponbare), which were transformed by Agrobacteria-mediated transformation method as described by Lin and Zhang ((2005) Plant Cell Rep. 23:540-547). The transgenic lines generated were developed and the transgenic seeds were harvested to form the rice activation tagging population.

Late flowering tagging lines (ATLs) were confirmed in repeated field experiments and their T-DNA insertion loci were determined. The T-DNA insertion loci in the ATLs were determined by Reverse-PCR or Southern-by-Sequencing method (Zastrow-Hayes G. M. et al. (2015), The Plant Genome, 8:1-15). The genes near by the left border and right border of the T-DNA were cloned and the functional genes were recapitulated by field screens. Only the recapitulated functional genes are showed herein. Based on LOC IDs and the corresponding gene sequences of these genes shown in Table 2, primers were designed for cloning the rice late flowering genes OsHIS (use SEQ ID NOs: 28 and 29), OsDN-FTG1 (use SEQ ID NOs: 30 and 31), OsWRKY76 (use SEQ ID NOs: 32 and 33), OsMYB77 (use SEQ ID NOs: 34 and 35), OsDN-FTG2 (use SEQ ID NOs: 36 and 37), OsENA1 (use SEQ ID NOs: 38 and 39), OsGRF1 (use SEQ ID NOs: 40 and 41), OsHIP14 (use SEQ ID NOs: 42 and 43), and OsDN-FTG3 (use SEQ ID NOs: 44 and 45).

TABLE 2
Rice gene names, Gene IDs (from TIGR) and Construct IDs

| Gene name | LOC ID | Construct ID |
|---|---|---|
| OsHIS | LOC_Os03g14669.2 | DP1492 |
| OsDN-FTG1 | LOC_Os01g04010.1 | DP1120 |
| OsWRKY76 | LOC_Os09g25060.1 | DP1189 |
| OSMYB77 | LOC_Os06g43090.1 | DP0207 |
| OsDN-FTG2 | LOC_Os03g30680.1 | DP0683 |
| OsENA1 | LOC_Os01g43080.1 | DP1438 |
| OsGRF1 | LOC_Os04g51190.1 | DP1707 |
| OsHIP14 | LOC_Os04g55510.1 | DP0696 |
| OsDN-FTG3 | LOC_Os03g61070.1 | DP2088 |

PCR amplified products were extracted after the agarose gel electrophoresis using a column kit and then ligated with TA cloning vectors. The sequences and orientation in these constructs were confirmed by sequencing. Each gene was cloned into a plant binary construct.

### Example 2

### Transformation and Gene Expression Analysis of Transgenic Rice Lines

Zhonghua 11 (Oryza sativa L.) were transformed with either a vector prepared in Example 1 or an empty vector (DP0158) by Agrobacteria-mediated transformation as described by Lin and Zhang ((2005) Plant Cell Rep. 23:540-547). Transgenic seedlings (T₀) generated in the transformation laboratory were transplanted in field to get T₁ seeds. The T₁ and subsequent T₂ seeds were screened to confirm transformation and positively identified transgenic seeds were used in the following trait screens.

The gene expression levels in the leaves of the transgenic rice plants were determined by RT-PCR. Primers were designed for the RT-PCR analyses of OsHIS (use SEQ ID NOs: 46 and 47), OsDN-FTG1 (use SEQ ID NOs: 48 and 49), OsWRKY76 (use SEQ ID NOs: 50 and 51), OsMYB77 (use SEQ ID NOs: 52 and 53), OsDN-FTG2 (use SEQ ID NOs: 54 and 55), OsENA1 (use SEQ ID NOs: 56 and 57), and OsDN-FTG3 (use SEQ ID NOs: 58 and 59) genes in the over-expression transgenic rice. The level of expression in ZH11-TC (tissue cultured ZH11 rice) was set at 1.00, and the expression levels in the DP1492, DP1120, DP1189, DP0207, DP0683, DP1438, and DP2088-transgenic rice plants were compared to ZH11-TC. Gene expression was normalized based on the EF-1α mRNA levels, and the results from the gene expression analysis are provided in Table 3 below.

TABLE 3
Relative Expression Level Fold
Increase in Transgenic Rice Plants

| | | Relative Expression |
|---|---|---|
| Gene name | Construct ID | Level Fold Increase |
| OsHIS | DP1492 | from 1.30 to 10.22 |
| OsDN-FTG1 | DP1120 | from 1.59 to 6.66 |
| OsWRKY76 | DP1189 | from 0.86 to 421.94 |
| OsMYB77 | DP0207 | from 0.37 to 71.79 |
| OsDN-FTG2 | DP0683 | from 141.14 to 966.56 |
| OsENA1 | DP1438 | from 1.39 to 273.64 |
| OsDN-FTG3 | DP2088 | from 1.43 to 21.11 |

### Example 3

### Characterization of the Transgenic Rice Plants

The transgenic rice plants from Example 2 and ZH11-TC and DP0158 rice plants were tested for in a Beijing field (40°13′N), a Hainan field (18°30′N), or a Changsha field (28°11′N) and the phenotypes were recorded during the plant growth.

Late flowering validation. The germinated seeds were planted in a seedbed field, and at 3-leaf stage, the seedlings were transplanted into field. Ten plants from each line were planted in one row. ZH11-TC (tissue cultured Zhonghua 11) was planted nearby the line in the same block and used as a control. The rice plants were managed by normal practice using pesticides and fertilizers. Plant phenotypes including heading date were observed and recorded during the experiments.

The heading dates include the first heading date and the 50% heading date. The first heading date is the date when the first panicle, usually the main stem panicle, headed out of the sheath of the flag leaf; and the 50% heading date is the date when 50% young panicles head out of the sheath of the flag leaf for plants in one row. The maturity date is the date when 90% glume, grain spikelet axis or vice glume become yellow from appearance. First Heading Time is defined as the days from sowing the seeds to the first heading date was calculated for each plant and statistically analyzed by t-test.

The results from these studies are provided in Table 4, which provides the combined data of the transgenic lines for each of the constructs.

TABLE 4
Flowering/Heading Time Characterizations
of the Transgenic Rice Plants

| | | | First Heading |
|---|---|---|---|
| NO | Location | Construct ID | Time (days) |
| 1 | Beijing | ZH11-TC | 98.78 ± 3.88 |
| | | DP1492 | 123.20 ± 3.11 ^{a} |
| 2 | Changsha | ZH11-TC | 69.28 ± 1.27 |
| | | DP1492 | 86.58 ± 1.32 ^{a} |
| 3 | Hainan | ZH11-TC | 57.36 ± 5.40 |
| | | DP1120 | 80.53 ± 9.02 ^{a} |
| 4 | Changsha | ZH11-TC | 70.20 ± 1.73 |
| | | DP1120 | 72.61 ± 2.50 ^{a} |
| 5 | Beijing | ZH11-TC | 98.78 ± 3.88 |
| | | DP1189 | 106.87 ± 3.03 ^{a} |
| 6 | Changsha | ZH11-TC | 69.28 ± 1.27 |
| | | DP1189 | 74.71 ± 4.40 ^{a} |
| 7 | Beijing | ZH11-TC | 98.78 ± 3.88 |
| | | DP0207 | 108.35 ± 4.69 ^{a} |
| 8 | Hainan | ZH11-TC | 60.11 ± 4.09 |
| | | DP0207 | 68.40 ± 5.08 ^{a} |
| 9 | Changsha | ZH11-TC | 70.20 ± 1.73 |
| | | DP0207 | 76.63 ± 3.44 ^{a} |
| 10 | Beijing | ZH11-TC | 98.78 ± 3.88 |
| | | DP0683 | 111.69 ± 3.79 ^{a} |
| 11 | Changsha | ZH11-TC | 69.28 ± 1.27 |
| | | DP0683 | 80.82 ± 1.50 ^{a} |
| 12 | Hainan | ZH11-TC | 60.11 ± 4.09 |
| | | DP0683 | 69.66 ± 4.44 ^{a} |
| 13 | Beijing | ZH11-TC | 98.78 ± 3.88 |
| | | DP1438 | 106.24 ± 2.46 ^{a} |
| 14 | Changsha | ZH11-TC | 98.78 ± 3.88 |
| | | DP1438 | 71.54 ± 0.81 ^{a} |
| 15 | Hainan | ZH11-TC | 60.11 ± 4.09 |
| | | DP1438 | 68.22 ± 4.81 ^{a} |
| 16 | Beijing | ZH11-TC | 98.78 ± 3.88 |
| | | DP1707 | 108.79 ± 11.23 ^{a} |
| 17 | Hainan | ZH11-TC | 60.11 ± 4.09 |
| | | DP1707 | 65.47 ± 4.10 ^{a} |
| 18 | Hainan | ZH11-TC | 60.11 ± 4.09 |
| | | DP0696 | 69.38 ± 5.19 ^{a} |
| 19 | Changsha | ZH11-TC | 70.20 ± 1.73 |
| | | DP0696 | 72.64 ± 2.45 ^{a} |
| 20 | Beijing | ZH11-TC | 98.78 ± 3.88 |
| | | DP2088 | 106.89 ± 3.66 ^{a} |
| 21 | Changsha | ZH11-TC | 69.28 ± 1.27 |
| | | DP2088 | 102.16 ± 6.61 ^{a} |

a P ≤ 0.01 compared to ZH11-TC control.

DP1492-transgenic rice plants showed late flowering at the T1 generation in a Beijing field, 15 transgenic events were planted and 14 of the events showed late flowering, the average first heading time of these 14 lines was 17.0 days later than that of the ZH11-TC control. To further investigate the flowering trait of DP1492 transgenic rice plants and to investigate whether the temperature or photoperiod affect the heading date or flowering time in rice, T1 seeds were planted in different locations or environments: Beijing (40°13′N) and Changsha (28°11′N). Twelve DP1492 overexpression rice lines were tested in the Beijing field. As shown in Table 4, The first heading time of the 12 lines was significantly later (P<0.01) than that of the ZH11-TC control, the average heading days of these 12 lines was 24.4 days later than that of the ZH11-TC control. Fourteen DP1492 overexpression rice lines were tested in the Changsha field. As shown in Table 4, the first heading time of the 14 lines was significantly later (P<0.01) than that of the ZH11-TC control, the average heading days of these 14 lines was 17.3 days later than that of the ZH11-TC control. These data show that OsHIS is a late flowering gene.

DP1120-transgenic rice plants showed late flowering at the T0 generation in a Hainan field, 60 T0 transgenic plants were planted and all the plants showed late flowering, the average first heading time of these 60 plants was 35 days later than that of the ZH11-TC control. To further investigate the flowering trait of DP1120 transgenic rice plants and to investigate whether the temperature or photoperiod affect the heading date or flowering time in rice, T1 seeds were planted in different locations or environments: Hainan (18°30′N) and Changsha (28°11′N). Five DP1120 transgenic rice lines were tested in the Hainan field. As shown in Table 4, the first heading time of the 5 lines was significantly later (P<0.01) than that of the ZH11-TC control, and the average first heading time of these 5 lines was 23.2 days later than that of the ZH11-TC control. Five DP1022 transgenic rice lines were tested in the Changsha field, the first heading time of the 5 lines was significantly later (P<0.01) than that of the ZH11-TC control, and the average first heading time of these 5 lines was 2.4 days later than that of ZH11-TC control. These data show that OsDN-FTG1 is a late flowering gene.

DP1189-transgenic rice plants showed late flowering in the T0 generation in a Hainan field, 59 T0 transgenic events were planted and all the plants showed late flowering, the average first heading time of these 59 plants was 10.0 days later than that of the ZH11-TC control. To further investigate the flowering trait of DP1189-transgenic rice plants and to investigate whether the temperature or photoperiod affect the heading date or flowering time in rice, T1 seeds were planted in different locations or environments: Beijing (40°13′N) and Changsha (28°11′N). Thirteen DP1189-transgenic rice lines were tested in the Beijing field. As shown in Table 4, the heading days of 13 lines was significantly later (P<0.01) than that of the ZH11-TC control, the average first heading time of these 13 lines is 8.1 days later than that of the ZH11-TC control. These 13 DP1189-transgenic rice lines were also tested in the Changsha field, the heading days of 13 lines was significantly later (P<0.01) than that of the ZH11-TC control, the average first heading time of these 13 lines was 6.4 days later than that of the ZH11-TC control. These results show that OsWRKY76 is a late flowering gene.

DP0207-transgenic rice plants showed late flowering at the T1 generation in a Beijing field, 8 T1 transgenic events were planted and 5 events showed late flowering, the average first heading time of these 5 plants was 20.0 days later than that of the ZH11-TC control. To further investigate the flowering trait of DP0207 transgenic rice plants and to investigate whether the temperature or photoperiod affect the heading date or flowering time in rice, T1 seeds were planted in different locations or environments:

Beijing (40°13′N), Changsha (28°11′N) and Hainan (18°30′N). Six DP0207-transgenic rice lines were tested in the Beijing field. As shown in Table 4, the first heading time of these 6 lines was significantly later (P<0.01) than that of the ZH11-TC control, and the average first heading time of these 6 lines is 9.6 days later than that of the ZH11-TC control. These 6 DP0207-transgenic rice lines were also tested in the Hainan field, the first heading time of 6 lines was significantly later (P<0.01) than that of the ZH11-TC control, and the average heading days of these 6 lines was 8.3 days later than that of the ZH11-TC control. Seven DP0207 transgenic rice lines were tested in the Changsha field, the first heading time of 7 lines was significantly later (P<0.01) than that of ZH11-TC control, and the average heading days of these 7 lines was 6.4 days later than that of the ZH11-TC control. These results demonstrate that OsMYB77 is a late flowering gene.

DP0683-transgenic rice plants showed late flowering at the T0 generation in a Beijing field, 74 T0 transgenic plants were planted and all the plants showed late flowering, the average first heading time of these 74 plants was 10.0 days later than that of the ZH11-TC control. To further investigate the flowering trait of DP0683 transgenic rice plants and to investigate whether the temperature or photoperiod affect the heading date or flowering time in rice, T1 seeds were planted in different locations or environments:

Beijing (40°13′N), Changsha (28°11′N) and Hainan (18°30′N). Fourteen DP0683 transgenic rice lines were tested in the Beijing field. As shown in Table 4, the first heading time of the 14 lines was significantly later (P<0.01) than that of the ZH11-TC control, and the average first heading time of these 14 lines was 12.9 days later than that of the ZH11-TC control. These 14 DP0683 transgenic rice lines were also tested in the Changsha field, and the first heading time of 14 lines was significantly later (P<0.01) than that of ZH11-TC control, and the average first heading time of these 14 lines was 11.5 days later than that of the ZH11-TC control. These 14 DP0683 transgenic rice lines were tested in the Hainan field, the heading days of 14 lines was significantly later (P<0.01) than that of ZH11-TC control, and the average first heading time of these 14 lines was 9.6 days later than that of the ZH11-TC control. These data show that OsDN-FTG2 is a late flowering gene.

DP1438 transgenic rice plants showed late flowering at T1 generation in a Hainan field, 13 T1 transgenic events were planted and all the events showed late flowering, the average first heading time of these 13 events was 5.0 days later than that of the ZH11-TC control. To further investigate the flowering trait of DP1438 transgenic rice plants and to investigate whether the temperature or photoperiod affect the heading date or flowering time in rice, T1 seeds were planted in different locations or environments: Beijing (40°13′N) and Hainan (18°30′N). Thirteen DP1438 transgenic rice lines were tested in the Beijing field. As shown in Table 4, the first heading time of 13 lines was significantly later (P<0.01) than that of ZH11-TC control, and the average first heading time of these 13 lines was 9.3 days later than that of ZH11-TC control. Ten DP1438 transgenic rice lines were also tested in the Hainan field, the heading days of 10 lines was significantly later (P<0.01) than that of the ZH11-TC control, and the average first heading time of these 10 lines was 8.1 days later than that of the ZH11-TC control. These data show that OsENA1 is a late flowering gene.

DP1707 transgenic rice plants showed late flowering at the T0 generation in a Hainan field, 21 T0 transgenic plants were planted and 10 plants showed late flowering. To further investigate the flowering trait of DP1438 transgenic rice plants and to investigate whether the temperature or photoperiod affect the heading date or flowering time in rice, T1 seeds were planted in different locations or environments: Beijing (40°13′N) and Hainan (18°30′N). Five DP1707 transgenic rice lines were tested in the Beijing field. As shown in Table 4, the first heading time of 5 lines was significantly later (P<0.01) than that of the ZH11-TC control, and the average first heading time of these 5 lines was 10.0 days later than that of the ZH11-TC control. These 5 DP1707 transgenic rice lines were tested in the Hainan field, the first heading time of 5 lines showed significantly later (P<0.01) than that of ZH11-TC control, and the average first heading time of these 5 lines was 5.4 days later than that of the ZH11-TC control. These data show that OsGRF1 is a late flowering gene.

DP0696 transgenic rice plants showed late flowering at the T0 generation in a Beijing field, 57 T0 transgenic plants were planted and all the plants showed late flowering, the average heading days of these 57 plants was 10 days later than that of the ZH11-TC control. To further investigate the flowering trait of DP0696 transgenic rice plants and to investigate whether the temperature or photoperiod affect the heading date or flowering time in rice, T1 seeds were planted in different locations or environments: Hainan (18°30′N) and Changsha (28°11′N). Fifteen DP0696-transgenic rice lines were tested in the Beijing field. As shown in Table 4, the first heading time of 15 lines was significantly later (P<0.01) than that of the ZH11-TC control, and the average first heading time of these 15 lines was 9.3 days later than that of the ZH11-TC control. Fifteen DP0696 transgenic rice lines were tested in Changsha field, the first heading time of the 15 lines was significantly later (P<0.01) than that of the ZH11-TC control, and the average first heading time of these 15 lines was 2.4 days later than that of the ZH11-TC control. These results show that OsHIP14 is a late flowering gene.

DP2088 transgenic rice plants showed late flowering at the T0 generation in a Beijing field, 50 T0 transgenic plants were planted and 33 plants showed late flowering, the average first heading time of these 33 plants was about 10 to 15 days later than that of the ZH11-TC control. To further investigate the flowering trait of DP2088 transgenic rice plants and to investigate whether the temperature or photoperiod affect the heading date or flowering time in rice, T1 seeds were planted in different locations or environments: Beijing (40°13′N) and Changsha (28°11′N). Thirteen DP2088 transgenic rice lines were tested in the Beijing field. As shown in Table 4, the first heading time of the 13 lines was significantly later (P<0.01) than that of the ZH11-TC control, and the average first heading time of these 13 lines was 8.1 days later than that of the ZH11-TC control. These 13 DP2088 transgenic rice lines were also tested in the Changsha field, the first heading time of the 13 lines was significantly later (P<0.01) than that of the ZH11-TC control, and the average first heading time of these 13 lines was 32.3 days later than that of the ZH11-TC control. These data show that OsDN-FTG3 is a late flowering gene.

Taken together, these results indicate that over-expression of OsHIS, OsDN-FTG1, OsWRKY76, OsMYB77, OsDN-FTG2, OsENA1, OsGRF1, OsHIP14 and OsDN-FTG3 delayed flowering time compared to control plants.

### Example 4

### Transformation and Evaluation of Maize with Rice Late Flowering Genes

Maize plants will be transformed with one of the polynucleotides encoding the polypeptides described herein or a corresponding homolog from maize, Arabidopsis, or other species. Expression of the gene in the maize transformation vector can be under control of a constitutive promoter such as the maize ubiquitin promoter (Christensen et al. (1989) Plant Mol. Biol. 12:619-632 and Christensen et al. (1992) Plant Mol. Biol. 18:675-689) or under control of another promoter, such as a stress-responsive promoter or a tissue-preferred promoter. The recombinant DNA construct can be introduced into maize cells by particle bombardment substantially as described in International Patent Publication WO 2009/006276. Alternatively, maize plants can be transformed with the recombinant DNA construct by Agrobacterium-mediated transformation substantially as described by Zhao et al. in Meth. Mol. Biol. 318:315-323 (2006) and in Zhao et al., Mol. Breed. 8:323-333 (2001) and U.S. Pat. No. 5,981,840 issued Nov. 9, 1999.

Progeny of the regenerated plants, such as T₁ plants, can be subjected to field tests. The heading time and maturity can be measured at multiple locations. Significant alternations in flowering time and/or maturity relative to a control, will be considered evidence that the gene functions in maize.

### Example 5

### Laboratory Screening of Rice Late Flowering Genes in Arabidopsis

To understand whether rice late flowering genes can improve dicot plants' late flowering or other traits, the rice expression vectors described herein can be transformed into Arabidopsis (Columbia) using floral dip method by Agrobacterium mediated transformation procedure and transgenic plants were identified (Clough, S. T. and Bent, A. F. (1998) The Plant Journal 16, 735-743; Zhang, X. et al. (2006) Nature Protocols 1: 641-646).

Progeny of the regenerated plants, such as T₁ plants, can be subjected to field tests. The heading time and maturity can be measured. Significant alternations in flowering time and/or maturity relative to a control, will be considered evidence that the gene functions in Arabidopsis.

1-6. (canceled)

7. A modified plant or modified seed comprising an increased expression of at least one polynucleotide encoding a polypeptide comprising an amino acid sequence of at least 90% sequence identity to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, wherein the modified plant or a plant grown from the modified seed exhibits delayed flowering time or maturity when compared to the control plant.

8. The modified plant or modified seed of claim 7, wherein the modified plant or modified seed comprises in its genome a recombinant DNA construct comprising a polynucleotide encoding a polypeptide with an amino acid sequence of at least 90% sequence identity to SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127 operably linked to at least one regulatory element.

9. The modified plant or modified seed of claim 7, wherein the modified plant or modified seed comprises a targeted genetic modification at a genomic locus comprising a polynucleotide sequence encoding a polypeptide with an amino acid sequence of at least 90% sequence identity to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127, thereby increasing expression of the polypeptide.

10. The modified plant or modified seed of claim 7, wherein said plant is selected from the group consisting of rice, maize, soybean, sunflower, sorghum, canola, wheat, alfalfa, cotton, barley, millet, sugar cane and switchgrass.

11. A method of delaying flowering time in a plant, comprising increasing the expression of at least one polynucleotide encoding a polypeptide comprising an amino acid sequence of at least 90% sequence identity to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

12. The method of claim 11, wherein the method comprises:
(a) expressing in a regenerable plant cell a recombinant DNA construct comprising a regulatory element operably liked to the polynucleotide sequence; and
(b) generating the plant, wherein the plant comprises in its genome the recombinant DNA construct.

13. The method of claim 11, wherein the method comprises:
(a) introducing in a regenerable plant cell a targeted genetic modification at a genomic locus that encodes a polypeptide comprising an amino acid sequence of at least 90% sequence identity compared to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and
(b) generating the plant, wherein the level and/or activity of the polypeptide is increased in the plant.

14. The method of claim 13, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

15. The method of claim 13, wherein the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 90% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

16. The method of claim 12, wherein the regulatory element is a heterologous promoter.

17-18. (canceled)

19. The method of claim 11, wherein said plant is selected from the group consisting of rice, maize, soybean, sunflower, sorghum, canola, wheat, alfalfa, cotton, barley, millet, sugar cane and switchgrass.

20. A method of accelerating flowering time in a plant, comprising decreasing the expression of at least one polynucleotide encoding a polypeptide comprising an amino acid sequence of at least 90% sequence identity to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

21. The method of claim 20, wherein the method comprises:
(a) introducing into a regenerable plant cell an RNAi construct targeting a polynucleotide encoding a polypeptide comprising an amino acid sequence of at least 90% sequence identity to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105,107, 109, 111, 113, 115, 117, 119, 121, 123, 125, or 127; and
(b) generating the plant, wherein the plant has decreased expression of the polynucleotide.

22. The method of claim 20, wherein the method comprises:
(a) introducing in a regenerable plant cell a targeted genetic modification at a genomic locus that encodes a polypeptide comprising an amino acid sequence of at least 90% sequence identity compared to SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127; and
(b) generating the plant, wherein the level and/or activity of the polypeptide is decreased in the plant.

23. The method of claim 22, wherein the targeted genetic modification is introduced using a genome modification technique selected from the group consisting of a polynucleotide-guided endonuclease, CRISPR-Cas endonucleases, base editing deaminases, a zinc finger nuclease, a transcription activator-like effector nuclease (TALEN), engineered site-specific meganucleases, or Argonaute.

24. The method of claim 22, wherein the targeted genetic modification is present in (a) the coding region; (b) a non-coding region; (c) a regulatory sequence; (d) an untranslated region; or (e) any combination of (a)-(d) of the genomic locus that encodes a polypeptide comprising an amino acid sequence that is at least 90% identical to an amino acid sequence selected from the group consisting of SEQ ID NOs: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

25. The method of claim 21, wherein said plant is selected from the group consisting of rice, maize, soybean, sunflower, sorghum, canola, wheat, alfalfa, cotton, barley, millet, sugar cane and switchgrass.

26. The modified plant or modified seed of claim 7, wherein the polynucleotide comprises a nucleotide sequence of SEQ ID NO: 1, SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23. SEQ ID NO: 25, SEQ ID NO: 26, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, or SEQ ID NO: 126.

27. The modified plant or modified seed of claim 7, wherein the encoded polypeptide comprises an amino acid sequence of SEQ ID NO: 3, 6, 9, 12, 15, 18, 21, 24, 27, 61, 63, 65. 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, or 127.

28. The modified plant or modified seed of claim 8, wherein the regulatory element is a heterologous promoter.


SEQUENCE LISTING (127 sequences)

SEQ ID NO: 1 - DNA, 874 bp, Oryza sativa
acacacagctacaaatcgactgtaattaaggtacgtatatataggtgacaatggacaaccagcagctaccctacgccggtcagccggcggccgcaggcgccggagccccggtgccgggcgtgcctggcgcgggcgggccgccggcggtgccgcaccaccacctgctccagcagcagcaggcgcagctgcaggcgttctgggcgtaccagcggcaggaggcggagcgcgcgtcggcgtcggacttcaagaaccaccagctgccgctggcgcggatcaagaagatcatgaaggcggacgaggacgtgcgcatgatctcggcggaggcgcccgtgctgttcgccaaggcgtgcgagctcttcatcctggagctcaccatccgctcgtggctgcacgccgaggagaacaagcgccgcaccctgcagcgcaacgacgtcgccgccgccatcgcgcgcaccgacgtgttcgacttcctcgtcgacatcgtgccgcgggaggaggccaaggaggagcccggcagcgcgctcgggttcgcggcgggagggcccgccggcgccgttggagcggccggccccgccgcggggctgccgtactactacccgccgatggggcagccggcgccgatgatgccggcgtggcatgttccggcgtgggacccggcgtggcagcaaggagcagcgccggatgtggaccagggcgccgccggcagcttcagcgaggaagggcagcaaggttttgcaggccatggcggtgcggcagctagcttccctcctgcacctccaagctccgaatagtgatgatccatatggttccatgcatgcatcgctgaggtgctagctagctactatagctgctcaaatcaaatgctcaatgtgtc

SEQ ID NO: 2 - DNA, 741 bp, Oryza sativa
atggacaaccagcagctaccctacgccggtcagccggcggccgcaggcgccggagccccggtgccgggcgtgcctggcgcgggcgggccgccggcggtgccgcaccaccacctgctccagcagcagcaggcgcagctgcaggcgttctgggcgtaccagcggcaggaggcggagcgcgcgtcggcgtcggacttcaagaaccaccagctgccgctggcgcggatcaagaagatcatgaaggcggacgaggacgtgcgcatgatctcggcggaggcgcccgtgctgttcgccaaggcgtgcgagctcttcatcctggagctcaccatccgctcgtggctgcacgccgaggagaacaagcgccgcaccctgcagcgcaacgacgtcgccgccgccatcgcgcgcaccgacgtgttcgacttcctcgtcgacatcgtgccgcgggaggaggccaaggaggagcccggcagcgcgctcgggttcgcggcgggagggcccgccggcgccgttggagcggccggccccgccgcggggctgccgtactactacccgccgatggggcagccggcgccgatgatgccggcgtggcatgttccggcgtgggacccggcgtggcagcaaggagcagcgccggatgtggaccagggcgccgccggcagcttcagcgaggaagggcagcaaggttttgcaggccatggcggtgcggcagctagcttccctcctgcacctccaagctccgaatag

SEQ ID NO: 3 - protein, 246 aa, Oryza sativa
MDNQQLPYAGQPAAAGAGAPVPGVPGAGGPPAVPHHHLLQQQQAQLQAFWAYQRQEAERASASDFKNHQLPLARIKKIMKADEDVRMISAEAPVLFAKACELFILELTIRSWLHAEENKRRTLQRNDVAAAIARTDVFDFLVDIVPREEAKEEPGSALGFAAGGPAGAVGAAGPAAGLPYYYPPMGQPAPMMPAWHVPAWDPAWQQGAAPDVDQGAAGSFSEEGQQGFAGHGGAAASFPPAPPSSE

SEQ ID NO: 4 - DNA, 673 bp, Oryza sativa
acaatctctctatctctctctctcttccttacgcggcaaaacacaccgtcgtccatggacagttctaataacaacaacaacaacaagagggcgcgcgacgccgaggatgaggccgacgaggccaagaggctgcgggcggaggacctgctcgacatgctcgacgatgataccgacgccgggggcgccgccggcgacctggcgtccgtcatgcggagcttcgaggaggagattgttgctggggatgtcgccggggacgttgcccccacgacgcagcccgagctcgggttccttctcgaggcctccgacgacgagctcggcctgccgcccgccaccgcgtcctcgtcggaggaggaggccggggctggggagcccgaggatgccatcgggttcggcggacagatctgggggttcgaggacgagattggcggaggcggctacgctggcttcgccctcacctcgccggaggcggtcgccgccgccgccgccgcggcggagtgggacgacgacggcttcgacgccggcttgttcggcttcggcgacgaggtctcggcgcttcgccacgagaccatgccggccgtctgatgggcctgggccgaagtggattagtttttttttattttcctcgttttttttaaatttggctaaccttagctctacccttgtttaaccttgg

SEQ ID NO: 5 - DNA, 528 bp, Oryza sativa
atggacagttctaataacaacaacaacaacaagagggcgcgcgacgccgaggatgaggccgacgaggccaagaggctgcgggcggaggacctgctcgacatgctcgacgatgataccgacgccgggggcgccgccggcgacctggcgtccgtcatgcggagcttcgaggaggagattgttgctggggatgtcgccggggacgttgcccccacgacgcagcccgagctcgggttccttctcgaggcctccgacgacgagctcggcctgccgcccgccaccgcgtcctcgtcggaggaggaggccggggctggggagcccgaggatgccatcgggttcggcggacagatctgggggttcgaggacgagattggcggaggcggctacgctggcttcgccctcacctcgccggaggcggtcgccgccgccgccgccgcggcggagtgggacgacgacggcttcgacgccggcttgttcggcttcggcgacgaggtctcggcgcttcgccacgagaccatgccggccgtctga

SEQ ID NO: 6 - protein, 175 aa, Oryza sativa
MDSSNNNNNNKRARDAEDEADEAKRLRAEDLLDMLDDDTDAGGAAGDLASVMRSFEEEIVAGDVAGDVAPTTQPELGFLLEASDDELGLPPATASSSEEEAGAGEPEDAIGFGGQIWGFEDEIGGGGYAGFALTSPEAVAAAAAAAEWDDDGFDAGLFGFGDEVSALRHETMPAV

SEQ ID NO: 7 - DNA, 1244 bp, Oryza sativa
cctgtttctgttttgattactcgagctccagagcacagcagcagagagacgcgagctgttcgtggtcgtcgtcgtcgtcgatggacgcggcgtggcgcggcggcgttggctgctcgccggtctgcctcgacctctgcgtcgggctgtcgccggtgcgggagccgtcggcggcgaggcacgagctgcttgaccggccggccggctgccgcggcggtggggattccaagtcgatgaccaatgacgaggtgaggtgttcttgattgattgattgattgattgattgatcggttgattgctcccgcggattcggttcttgcttggttttgatcgttggaaatgtggggggattccttcaggcgaagatcctcgaggcgaaggtcactcagatgagcgaggagaatcggcggctgaccgaggtgatcgcccgcctgtacggcggccaaatcgcgcggctcggcctcgacggctcggcctcgccgccgcggccggtgtcgccgttatcgggcaagaagaggagcagggagagcatggagacggcgaattcctgcgacgccaacagcaacaggcatcagggcggcgacgccgaccacgccgagagcttcgccgccgacgatggcacctgccggaggatcaaggtcagccgggtgtgcaggcggatcgacccgtcggacacctccctggtggtcaaggacgggtaccaatggcggaagtacgggcagaaggtgacgcgcgacaacccgtcgccgagggcctacttccggtgcgccttcgcgccgtcgtgcccggtgaagaagaaggtgcagcggagcgcggaggacagctcgctggtggtggcgacgtacgagggcgagcacaaccacccgcacccgtctccgcgcgccggcgagctcccggcggcggtggggggggccggtggctcgctgccgtgctccatctccatcaactcctccggcccgaccatcacgctcgacctcaccaagaacgggggagccgtgcaggtggtcgaggcggcgcatccgccgccgccgccggacctcaaggaggtgtgccgggaggtcgcgtcgccggagttccggaccgcgctggtggagcagatggccagcgcgctcaccagcgaccccaagttcaccggcgcgctcgccgcggcgatcctccagaagctgcccgaattctagcttcctttacaattctccaattcttcttacaggaaaaaacatagaggcgcatttcaatagagattagag

SEQ ID NO: 8 - DNA, 984 bp, Oryza sativa
atggacgcggcgtggcgcggcggcgttggctgctcgccggtctgcctcgacctctgcgtcgggctgtcgccggtgcgggagccgtcggcggcgaggcacgagctgcttgaccggccggccggctgccgcggcggtggggattccaagtcgatgaccaatgacgaggcgaagatcctcgaggcgaaggtcactcagatgagcgaggagaatcggcggctgaccgaggtgatcgcccgcctgtacggcggccaaatcgcgcggctcggcctcgacggctcggcctcgccgccgcggccggtgtcgccgttatcgggcaagaagaggagcagggagagcatggagacggcgaattcctgcgacgccaacagcaacaggcatcagggcggcgacgccgaccacgccgagagcttcgccgccgacgatggcacctgccggaggatcaaggtcagccgggtgtgcaggcggatcgacccgtcggacacctccctggtggtcaaggacgggtaccaatggcggaagtacgggcagaaggtgacgcgcgacaacccgtcgccgagggcctacttccggtgcgccttcgcgccgtcgtgcccggtgaagaagaaggtgcagcggagcgcggaggacagctcgctggtggtggcgacgtacgagggcgagcacaaccacccgcacccgtctccgcgcgccggcgagctcccggcggcggtggggggggccggtggctcgctgccgtgctccatctccatcaactcctccggcccgaccatcacgctcgacctcaccaagaacgggggagccgtgcaggtggtcgaggcggcgcatccgccgccgccgccggacctcaaggaggtgtgccgggaggtcgcgtcgccggagttccggaccgcgctggtggagcagatggccagcgcgctcaccagcgaccccaagttcaccggcgcgctcgccgcggcgatcctccagaagctgcccgaattctag

SEQ ID NO: 9 - protein, 327 aa, Oryza sativa
MDAAWRGGVGCSPVCLDLCVGLSPVREPSAARHELLDRPAGCRGGGDSKSMTNDEAKILEAKVTQMSEENRRLTEVIARLYGGQIARLGLDGSASPPRPVSPLSGKKRSRESMETANSCDANSNRHQGGDADHAESFAADDGTCRRIKVSRVCRRIDPSDTSLVVKDGYQWRKYGQKVTRDNPSPRAYFRCAFAPSCPVKKKVQRSAEDSSLVVATYEGEHNHPHPSPRAGELPAAVGGAGGSLPCSISINSSGPTITLDLTKNGGAVQVVEAAHPPPPPDLKEVCREVASPEFRTALVEQMASALTSDPKFTGALAAAILQKLPEF

SEQ ID NO: 10 - DNA, 990 bp, Oryza sativa
actcgtcgtggtagtagtagtcttcttcgcgtgacgccatgggaggaggaggaggagttgaggcggattgcgacaggatcagggggccgtggagccccgaggaggacgaggcgctgcggcggctggtggagcggcacggcgcgaggaactggacggcgatcgggcgggagatccccgggaggtcggggaagtcgtgccggctgcggtggtgcaaccagctctcgccgcaggtggagcggcggccgttcaccgccgaggaggacgccaccatcctccgcgcacacgcgcggctcgggaacaggtgggccgccatcgcgcgcctcctccagggccgcaccgacaacgccgtgaagaaccactggaactgctccctcaagcgcaagctcgccgtcgccaccaccaccaccaccaccaccaccggcgccgccgccgcgccgggagtggtcgccgatgccgccgagctcgtcgagcggccgtgcaagcggttcagccccacgccggacagcccgtcggggtctggttccgggtcggaccgcagcgacctcagccacggcggcgggttcgggcagattttccggccggtggcgaggaccggcgcgttcgagcccgtcgactgcgccatcagccggcggcaggaggaggatcccttcacctcgctctccctctcgctccctgggacggatcagcggttcaaccacgacagcgcccacagccacttccaagaactcccgtcctccccctcgccgccaccaccacctccccccgccgccgccgcctcgacgacacagtacccgttcaccccggagttcgccgccgcgatgcaggagatgatccgcgccgaggtgcacaagtacatggcgagcgtcggcgtccgcgccgggtgcggcgacgccggcggtgccgacctccacatgccgcagctggtggagggcgtcatgcgcgccgccgcggagcgcgtcgggaggatgcactgatcaaaaacctagcgtc

SEQ ID NO: 11 - DNA, 936 bp, Oryza sativa
atgggaggaggaggaggagttgaggcggattgcgacaggatcagggggccgtggagccccgaggaggacgaggcgctgcggcggctggtggagcggcacggcgcgaggaactggacggcgatcgggcgggagatccccgggaggtcggggaagtcgtgccggctgcggtggtgcaaccagctctcgccgcaggtggagcggcggccgttcaccgccgaggaggacgccaccatcctccgcgcacacgcgcggctcgggaacaggtgggccgccatcgcgcgcctcctccagggccgcaccgacaacgccgtgaagaaccactggaactgctccctcaagcgcaagctcgccgtcgccaccaccaccaccaccaccaccaccggcgccgccgccgcgccgggagtggtcgccgatgccgccgagctcgtcgagcggccgtgcaagcggttcagccccacgccggacagcccgtcggggtctggttccgggtcggaccgcagcgacctcagccacggcggcgggttcgggcagattttccggccggtggcgaggaccggcgcgttcgagcccgtcgactgcgccatcagccggcggcaggaggaggatcccttcacctcgctctccctctcgctccctgggacggatcagcggttcaaccacgacagcgcccacagccacttccaagaactcccgtcctccccctcgccgccaccaccacctccccccgccgccgccgcctcgacgacacagtacccgttcaccccggagttcgccgccgcgatgcaggagatgatccgcgccgaggtgcacaagtacatggcgagcgtcggcgtccgcgccgggtgcggcgacgccggcggtgccgacctccacatgccgcagctggtggagggcgtcatgcgcgccgccgcggagcgcgtcgggaggatgcactga

SEQ ID NO: 12 - protein, 311 aa, Oryza sativa
MGGGGGVEADCDRIRGPWSPEEDEALRRLVERHGARNWTAIGREIPGRSGKSCRLRWCNQLSPQVERRPFTAEEDATILRAHARLGNRWAAIARLLQGRTDNAVKNHWNCSLKRKLAVATTTTTTTTGAAAAPGVVADAAELVERPCKRFSPTPDSPSGSGSGSDRSDLSHGGGFGQIFRPVARTGAFEPVDCAISRRQEEDPFTSLSLSLPGTDQRFNHDSAHSHFQELPSSPSPPPPPPPAAAASTTQYPFTPEFAAAMQEMIRAEVHKYMASVGVRAGCGDAGGADLHMPQLVEGVMRAAAERVGRMH

SEQ ID NO: 13 - DNA, 928 bp, Oryza sativa
gcagagattgagagatgttggtgccggaggcggagctgcctgtgcagtcggcggcggcggcggcgccgatagactggatgtggtacacggtgcatctgacggtggaagagatcgagcgaatcacggcgagagttgaagcggttacgacggccctcgaggccatacgcccggcgctcgacatggccgtcgggctgctcggcgaggacatctacgccgccgagatcctcgacgactacatgctggccgccctcgtgcccgcaggcgcaggccaggccccgctcccggacgccaccctcgacgcggcggcgaggaccttcgccaccgtgtcctccggggcgccgctgctcccaggctccatccttgacgtcgggaacctcatctccgccgcgtacgacatcgtcgatcagccgccaccagatgcccccacccccgacgggctactcaacgatgccatcaccgatctccaagccgccttcgccgacgggggcctcctcaccaacgtccggaatcacttccaccactgcgccgcgtacctccatgttcaaccgatcgacgccgacccgacgtggacggcgtggaccgggcaagcgcagcaggccaactatttcgcgaccgacgcgttggcgatgctcaacgtcgtcgcctgggaggccatggacgcgatggagcttatccgctcccactgcctggttccgtcgccggagcgcaacgagcacatgagggagctcgagaggtgcctgctcacggccatcaagtacatcgacaaggcgattgcggcggtgggtctcgtgcacggcgaggtggagttgatggaccagacactccgtcaagccatccacgacgccaacatccctgctaatggctgggcttgaatccatgacgaaccgcaagcttccgcgaggatgacgagatcgatccccctttccac

SEQ ID NO: 14 - DNA, 858 bp, Oryza sativa
atgttggtgccggaggcggagctgcctgtgcagtcggcggcggcggcggcgccgatagactggatgtggtacacggtgcatctgacggtggaagagatcgagcgaatcacggcgagagttgaagcggttacgacggccctcgaggccatacgcccggcgctcgacatggccgtcgggctgctcggcgaggacatctacgccgccgagatcctcgacgactacatgctggccgccctcgtgcccgcaggcgcaggccaggccccgctcccggacgccaccctcgacgcggcggcgaggaccttcgccaccgtgtcctccggggcgccgctgctcccaggctccatccttgacgtcgggaacctcatctccgccgcgtacgacatcgtcgatcagccgccaccagatgcccccacccccgacgggctactcaacgatgccatcaccgatctccaagccgccttcgccgacgggggcctcctcaccaacgtccggaatcacttccaccactgcgccgcgtacctccatgttcaaccgatcgacgccgacccgacgtggacggcgtggaccgggcaagcgcagcaggccaactatttcgcgaccgacgcgttggcgatgctcaacgtcgtcgcctgggaggccatggacgcgatggagcttatccgctcccactgcctggttccgtcgccggagcgcaacgagcacatgagggagctcgagaggtgcctgctcacggccatcaagtacatcgacaaggcgattgcggcggtgggtctcgtgcacggcgaggtggagttgatggaccagacactccgtcaagccatccacgacgccaacatccctgctaatggctgggcttga

SEQ ID NO: 15 - protein, 285 aa, Oryza sativa
MLVPEAELPVQSAAAAAPIDWMWYTVHLTVEEIERITARVEAVTTALEAIRPALDMAVGLLGEDIYAAEILDDYMLAALVPAGAGQAPLPDATLDAAARTFATVSSGAPLLPGSILDVGNLISAAYDIVDQPPPDAPTPDGLLNDAITDLQAAFADGGLLTNVRNHFHHCAAYLHVQPIDADPTWTAWTGQAQQANYFATDALAMLNVVAWEAMDAMELIRSHCLVPSPERNEHMRELERCLLTAIKYIDKAIAAVGLVHGEVELMDQTLRQAIHDANIPANGWA

SEQ ID NO: 16 - DNA, 956 bp, Oryza sativa
cacccacccaccgatcatgttcgcgccgacgttcgcggtggccgccgctctggcgccgccgccgcctcgaggaggcggaggcggaggaggggagttcgaccacttcgtggtggtggacttcgaggcgacgtgcgagaggggcaggcggatctacccgcaggagatcatcgagttccccgcggtgctcgtggacgccgccacgggccgcctcgtgtccgcgttccgcgcctacgtccgcccgcgccaccacccgcggctcaccgacttctgccgcgagctcacggggatcgcccagggcgacgtcgacgccggggtgggcctcgccgaggcgctcctcaggcacgacgagtggctgcgtgcggccggggtcgtcgagggcggcgggcggttcgccgtcgtcacgtggggcgacgccgactgccgcaccatgctggagcaggagtgccggttcaagggcatcgcgaagccggcctacttcgaccggtgggtcgacctcagggtccacttcgaggcggcgttcggcggcggcgggcagcgggtgaagctgcaggaggcggtcagggcggcggggctggagtgggtggggcgcctgcactgcggcctcgacgacgcctgcaacacggcgcgcctcctcgtcgagctcttgcgccgcggcgtccccatctccatcaccggctcgctgccggccgcgccgccgccgcttgagcaagctcgtaagcagcagcagcagcaggagatgcagcagctgctcgtcccgtgcggcgcggcggtgtgctgctactgcggcgtggcgagcacgggaggggtgatggcgatgccggggtcgacgcagcggcggtgcttctacggctgcggcaactggacggcggtgtccggggcgacgtgccccttctttctcatgggcggcgtagtggattgtcctataaattagcaactctgtgctgggaattcttaca

SEQ ID NO: 17 - DNA, 915 bp, Oryza sativa
atgttcgcgccgacgttcgcggtggccgccgctctggcgccgccgccgcctcgaggaggcggaggcggaggaggggagttcgaccacttcgtggtggtggacttcgaggcgacgtgcgagaggggcaggcggatctacccgcaggagatcatcgagttccccgcggtgctcgtggacgccgccacgggccgcctcgtgtccgcgttccgcgcctacgtccgcccgcgccaccacccgcggctcaccgacttctgccgcgagctcacggggatcgcccagggcgacgtcgacgccggggtgggcctcgccgaggcgctcctcaggcacgacgagtggctgcgtgcggccggggtcgtcgagggcggcgggcggttcgccgtcgtcacgtggggcgacgccgactgccgcaccatgctggagcaggagtgccggttcaagggcatcgcgaagccggcctacttcgaccggtgggtcgacctcagggtccacttcgaggcggcgttcggcggcggcgggcagcgggtgaagctgcaggaggcggtcagggcggcggggctggagtgggtggggcgcctgcactgcggcctcgacgacgcctgcaacacggcgcgcctcctcgtcgagctcttgcgccgcggcgtccccatctccatcaccggctcgctgccggccgcgccgccgccgcttgagcaagctcgtaagcagcagcagcagcaggagatgcagcagctgctcgtcccgtgcggcgcggcggtgtgctgctactgcggcgtggcgagcacgggaggggtgatggcgatgccggggtcgacgcagcggcggtgcttctacggctgcggcaactggacggcggtgtccggggcgacgtgccccttctttctcatgggcggcgtagtggattgtcctataaattag

SEQ ID NO: 18 - protein, 304 aa, Oryza sativa
MFAPTFAVAAALAPPPPRGGGGGGGEFDHFVVVDFEATCERGRRIYPQEIIEFPAVLVDAATGRLVSAFRAYVRPRHHPRLTDFCRELTGIAQGDVDAGVGLAEALLRHDEWLRAAGVVEGGGRFAVVTWGDADCRTMLEQECRFKGIAKPAYFDRWVDLRVHFEAAFGGGGQRVKLQEAVRAAGLEWVGRLHCGLDDACNTARLLVELLRRGVPISITGSLPAAPPPLEQARKQQQQQEMQQLLVPCGAAVCCYCGVASTGGVMAMPGSTQRRCFYGCGNWTAVSGATCPFFLMGGVVDCPIN

SEQ ID NO: 19 - DNA, 1247 bp, Oryza sativa
gagtgtgtgtgtgtgtgagggagagagagatagcggcagcatatatggcgatgccctttgcctccctgtcgccggcagccgaccaccggccctccttcatcttccccttctgccgctcctcccctctctccgcggtcggggaggaggcgcagcagcacatgatgggcgcgaggtgggcggcggcggtggccaggccgccgcccttcacggcggcgcagtacgaggagctggagcagcaggcgctcatatacaagtacctcgtcgccggcgtgcccgtcccggcggatctcctcctccccatccgccgtggcctcgactcactcgcctcgcgcttctaccaccaccctgtccttggatacggttcctacttcggcaagaagctggacccggagcccggacggtgccggcgtacggacggcaagaagtggcggtgctccaaggaggccgcgccggactccaagtactgtgagcgacacatgcaccgcggccgcaaccgttcaagaaagcctgtggaagcgcagctcgtcgccccccactcgcagccccccgccacggcgccggccgccgccgtcacctccaccgccttccagaaccactcgctgtacccggcgattgctaatggcggcggcgccaacggaggcggtggtggtggtggcggtggcggcagcgcgcctggctcgttcgccttggggtctaatactcagctgcacatggacaatgctgcgtcttactcgactgttgctgctggtgccggaaacaaagatttcaggtattctgcttatggagtgagaccattggcagatgagcacagcccactcatcactggagctatggatacctctattgacaattcgtggtgcttgctgccttctcagacctccacattttcagtttcgagctaccctatgcttggaaatctgagtgagctggaccagaacaccatctgctcgctgccgaaggtggagagggagccattgtcattcttcgggagcgactatgtgaccgtcgactccgggaagcaggagaaccagacgctgcgcccctttttcgacgagtggccaaaggcaagggactcctggcctgatctagctgatgacaacagccttgccaccttctctgccactcagctctcgatctccattccaatggcaacctctgacttctcgaccaccagctcacgatcacacaacggtatatactcccgatgagtgaatatcatctggatccctacgtgtcaatgaagcgct

SEQ ID NO: 20 - DNA, 1164 bp, Oryza sativa
atggcgatgccctttgcctccctgtcgccggcagccgaccaccggccctccttcatcttccccttctgccgctcctcccctctctccgcggtcggggaggaggcgcagcagcacatgatgggcgcgaggtgggcggcggcggtggccaggccgccgcccttcacggcggcgcagtacgaggagctggagcagcaggcgctcatatacaagtacctcgtcgccggcgtgcccgtcccggcggatctcctcctccccatccgccgtggcctcgactcactcgcctcgcgcttctaccaccaccctgtccttggatacggttcctacttcggcaagaagctggacccggagcccggacggtgccggcgtacggacggcaagaagtggcggtgctccaaggaggccgcgccggactccaagtactgtgagcgacacatgcaccgcggccgcaaccgttcaagaaagcctgtggaagcgcagctcgtcgccccccactcgcagccccccgccacggcgccggccgccgccgtcacctccaccgccttccagaaccactcgctgtacccggcgattgctaatggcggcggcgccaacggaggcggtggtggtggtggcggtggcggcagcgcgcctggctcgttcgccttggggtctaatactcagctgcacatggacaatgctgcgtcttactcgactgttgctgctggtgccggaaacaaagatttcaggtattctgcttatggagtgagaccattggcagatgagcacagcccactcatcactggagctatggatacctctattgacaattcgtggtgcttgctgccttctcagacctccacattttcagtttcgagctaccctatgcttggaaatctgagtgagctggaccagaacaccatctgctcgctgccgaaggtggagagggagccattgtcattcttcgggagcgactatgtgaccgtcgactccgggaagcaggagaaccagacgctgcgcccctttttcgacgagtggccaaaggcaagggactcctggcctgatctagctgatgacaacagccttgccaccttctctgccactcagctctcgatctccattccaatggcaacctctgacttctcgaccaccagctcacgatcacacaacggtatatactcccggtaa

SEQ ID NO: 21 - protein, 387 aa, Oryza sativa
MAMPFASLSPAADHRPSFIFPFCRSSPLSAVGEEAQQHMMGARWAAAVARPPPFTAAQYEELEQQALIYKYLVAGVPVPADLLLPIRRGLDSLASRFYHHPVLGYGSYFGKKLDPEPGRCRRTDGKKWRCSKEAAPDSKYCERHMHRGRNRSRKPVEAQLVAPHSQPPATAPAAAVTSTAFQNHSLYPAIANGGGANGGGGGGGGGGSAPGSFALGSNTQLHMDNAASYSTVAAGAGNKDFRYSAYGVRPLADEHSPLITGAMDTSIDNSWCLLPSQTSTFSVSSYPMLGNLSELDQNTICSLPKVEREPLSFFGSDYVTVDSGKQENQTLRPFFDEWPKARDSWPDLADDNSLATFSATQLSISIPMATSDFSTTSSRSHNGIYSR

SEQ ID NO: 22 - DNA, 2061 bp, Oryza sativa
cagacatcaattgataaagagcacatcttcagtgatgcaaggacagaagaactctgttgagcagcttgctgatgtgtttggatttgaccatgcatccagttcaggaaaccctgtcatggatcaacaaggatattggaacaacattcttggttcagtggagtcacataaccttcaaggttatcaggtgaaccgtagtgatggaactattccctatggaaacggtgtgcatcaaaatggcacttttttaggtttttgggaatcaggcgaagcaagtgcaagtggcagttcactacattttgggggctctaatgagatcaaagctgagcaacgtaatatcggtggtggcctaaggattggtgaaaggcgcttggtagctgagcgcaacctttctttggataatgttgatataggccttaacatcaatggtaatgatctatctggtgaaaattcaaatgtgaatggtgcttcacaaggctctgaactacatggtggctgctctcatactggttcaaatggtcaagcctctgagctgagattacatccatacaggacattcatattaggtgcagatcaacctgagccttttaattctttgaatggcagtgaaaatcctttaggtgatttttccttgatgccagaaggcattgatcagcgaccaggcagttccctggatggccgccggctggcatgcaagaggaaaaatatagaaggagttaatgggcagtgctcggcaggtgctagcactagtttttcccacaggaacgatagtattttccataacattgcttcttcgagtcataatccctctcctagtacaaatttaccctctcctaattgtctgttggttccaagcactcttgatgaacaactcccgcgttatggagctactacagctggattgtcatctagtagctatgatcctagtggaggcaataacaattcaggaggctcacaaagaagttttcggccaagaactagcctggctcaacatattggcccctatggtgtgtggccatcttcaagtactatcagacattccaattcatggaatcatcagccacctccctttcagagttcatttgatgaaccaccggaggtaattccagtggtcagtagcctgaactttcaataccagcatccaatgaatgtcgttcctggcattccacagatgtcacaccggttcactggtccaggggcctcatcatcaagaacaggcaatttggagaacagaattattggtagtgaggaatttagcgcgaggaatgtagtggctaccagcttccctgatgcagttcctccggccgcactagacatgaggcatttgataccagaaccatctagttggaatgtagatggcagagctactaccattccaggaaatgttccttcttcatcgagagctaataccaattcgatggttaatccaccagcaggctctccatttattgcccatcaaaacttgcatagacgtaatcctcgtaatttgtcagaggagataagtcgtttatctggagctcttcgtggccatcagcacccacgcctaaggtccggttttctgttagaacgacaaggtgatggtgtttggggtgttccgttgtcaactaggagcagggaaggaagaagattaattgagatccggaacgcgcttgaaatgattcacagaggggaaaatgtaagatttgagtccattttctatggtggagtcgacattcatgacagacacagggatatgcgccttgacatagacaacatgtcttatgaggagctattggcactggaagaaagaataggcaatgttagcactggcctcagtgaggaagaagtgacaaagctcctaaagcaaaggaaattctcatcatggaggttggaagcatctgtggaagaagagccatgctgtatctgccaggaagagtatgttgatggggatgatctcgggacactggactgtggacatgacttccatgttggatgcgtcaggcaatggctggttgtgaagaacacctgtcccatatgcaaaaatactgctctgaagtcttagaaaaagcacagggcagtaaacac

SEQ ID NO: 23 - DNA, 2004 bp, Oryza sativa
atgcaaggacagaagaactctgttgagcagcttgctgatgtgtttggatttgaccatgcatccagttcaggaaaccctgtcatggatcaacaaggatattggaacaacattcttggttcagtggagtcacataaccttcaaggttatcaggtgaaccgtagtgatggaactattccctatggaaacggtgtgcatcaaaatggcacttttttaggtttttgggaatcaggcgaagcaagtgcaagtggcagttcactacattttgggggctctaatgagatcaaagctgagcaacgtaatatcggtggtggcctaaggattggtgaaaggcgcttggtagctgagcgcaacctttctttggataatgttgatataggccttaacatcaatggtaatgatctatctggtgaaaattcaaatgtgaatggtgcttcacaaggctctgaactacatggtggctgctctcatactggttcaaatggtcaagcctctgagctgagattacatccatacaggacattcatattaggtgcagatcaacctgagccttttaattctttgaatggcagtgaaaatcctttaggtgatttttccttgatgccagaaggcattgatcagcgaccaggcagttccctggatggccgccggctggcatgcaagaggaaaaatatagaaggagttaatgggcagtgctcggcaggtgctagcactagtttttcccacaggaacgatagtattttccataacattgcttcttcgagtcataatccctctcctagtacaaatttaccctctcctaattgtctgttggttccaagcactcttgatgaacaactcccgcgttatggagctactacagctggattgtcatctagtagctatgatcctagtggaggcaataacaattcaggaggctcacaaagaagttttcggccaagaactagcctggctcaacatattggcccctatggtgtgtggccatcttcaagtactatcagacattccaattcatggaatcatcagccacctccctttcagagttcatttgatgaaccaccggaggtaattccagtggtcagtagcctgaactttcaataccagcatccaatgaatgtcgttcctggcattccacagatgtcacaccggttcactggtccaggggcctcatcatcaagaacaggcaatttggagaacagaattattggtagtgaggaatttagcgcgaggaatgtagtggctaccagcttccctgatgcagttcctccggccgcactagacatgaggcatttgataccagaaccatctagttggaatgtagatggcagagctactaccattccaggaaatgttccttcttcatcgagagctaataccaattcgatggttaatccaccagcaggctctccatttattgcccatcaaaacttgcatagacgtaatcctcgtaatttgtcagaggagataagtcgtttatctggagctcttcgtggccatcagcacccacgcctaaggtccggttttctgttagaacgacaaggtgatggtgtttggggtgttccgttgtcaactaggagcagggaaggaagaagattaattgagatccggaacgcgcttgaaatgattcacagaggggaaaatgtaagatttgagtccattttctatggtggagtcgacattcatgacagacacagggatatgcgccttgacatagacaacatgtcttatgaggagctattggcactggaagaaagaataggcaatgttagcactggcctcagtgaggaagaagtgacaaagctcctaaagcaaaggaaattctcatcatggaggttggaagcatctgtggaagaagagccatgctgtatctgccaggaagagtatgttgatggggatgatctcgggacactggactgtggacatgacttccatgttggatgcgtcaggcaatggctggttgtgaagaacacctgtcccatatgcaaaaatactgctctgaagtcttag

SEQ ID NO: 24 - protein, 667 aa, Oryza sativa
MQGQKNSVEQLADVFGFDHASSSGNPVMDQQGYWNNILGSVESHNLQGYQVNRSDGTIPYGNGVHQNGTFLGFWESGEASASGSSLHFGGSNEIKAEQRNIGGGLRIGERRLVAERNLSLDNVDIGLNINGNDLSGENSNVNGASQGSELHGGCSHTGSNGQASELRLHPYRTFILGADQPEPFNSLNGSENPLGDFSLMPEGIDQRPGSSLDGRRLACKRKNIEGVNGQCSAGASTSFSHRNDSIFHNIASSSHNPSPSTNLPSPNCLLVPSTLDEQLPRYGATTAGLSSSSYDPSGGNNNSGGSQRSFRPRTSLAQHIGPYGVWPSSSTIRHSNSWNHQPPPFQSSFDEPPEVIPVVSSLNFQYQHPMNVVPGIPQMSHRFTGPGASSSRTGNLENRIIGSEEFSARNVVATSFPDAVPPAALDMRHLIPEPSSWNVDGRATTIPGNVPSSSRANTNSMVNPPAGSPFIAHQNLHRRNPRNLSEEISRLSGALRGHQHPRLRSGFLLERQGDGVWGVPLSTRSREGRRLIEIRNALEMIHRGENVRFESIFYGGVDIHDRHRDMRLDIDNMSYEELLALEERIGNVSTGLSEEEVTKLLKQRKFSSWRLEASVEEEPCCICQEEYVDGDDLGTLDCGHDFHVGCVRQWLVVKNTCPICKNTALKS

SEQ ID NO: 25 - DNA, 1084 bp, Oryza sativa
agcaaggaaatacccaaccccgagcgccaaaaccctctcctctcctcgccttgccgcgcccgcgcattcccgcaaacacctcgccggcggagcgcccccgagatgaagggttccgacctcccgcccggcggctccggccagaagccaccgggcggcccgccggggaacaggaagggcaagcgcggcgctgaggcgcctccggctacctcgtcgtcgacgccgacaaccgcgaggcggagtaagcgcctcgctggtgcgcctccggatcaccccgcggaagcagggccatcgtcgacgaacgcgaggcggagcagtcgcctcgctggtgcgcctcctgctacccccggggctgcggcggcagcgccgacctcgtcgtcgccgacagccgcaaggcggagcaatcgcctcgctggtgcgcctccggatccccccgcggctgtggcagcaccgccgacctcgtcgtcgccgacaaccgcgatgcggagcaatcgcctcgctggtgcgccttcggatccccccgcggctgcgacagcagggccgacctcgccatcgccgacaactgcgaggcggagcaatcgcctcgctggtgcgcctccggatcccccggcggctgcgacagcagggccgacctcgaaaaccgcgagacggagcaagcgcctcgctggtgaggctccggagacccccgtggaagcagggcagacctcgccgtcgtcgacaaccgcgaggcagagcaagcgcagcgctggtaagtctccggctatccccaagggatcggggcaaccctcgtcggccgagaagagcaagcgcaccgctgatgcgtcttcggctgaccccgcggaagcaaggccgtcgtcgctgtcgcccacaaccgcgccggtcaggactacggccgtgtcggtgtcggtgaggaaggcggccgaggggcagcggcggaccaccacctcggggcgcgggaccaccacctcggggcgcggggacgctgcggagcaggaggcgatgcgggaggcggtcctgtacgtgcgccgagagctctccgtcgtcgcgccggatgacctcacctcgccgcacaactagccgagggtgg

SEQ ID NO: 26 - DNA, 972 bp, Oryza sativa
atgaagggttccgacctcccgcccggcggctccggccagaagccaccgggcggcccgccggggaacaggaagggcaagcgcggcgctgaggcgcctccggctacctcgtcgtcgacgccgacaaccgcgaggcggagtaagcgcctcgctggtgcgcctccggatcaccccgcggaagcagggccatcgtcgacgaacgcgaggcggagcagtcgcctcgctggtgcgcctcctgctacccccggggctgcggcggcagcgccgacctcgtcgtcgccgacagccgcaaggcggagcaatcgcctcgctggtgcgcctccggatccccccgcggctgtggcagcaccgccgacctcgtcgtcgccgacaaccgcgatgcggagcaatcgcctcgctggtgcgccttcggatccccccgcggctgcgacagcagggccgacctcgccatcgccgacaactgcgaggcggagcaatcgcctcgctggtgcgcctccggatcccccggcggctgcgacagcagggccgacctcgaaaaccgcgagacggagcaagcgcctcgctggtgaggctccggagacccccgtggaagcagggcagacctcgccgtcgtcgacaaccgcgaggcagagcaagcgcagcgctggtaagtctccggctatccccaagggatcggggcaaccctcgtcggccgagaagagcaagcgcaccgctgatgcgtcttcggctgaccccgcggaagcaaggccgtcgtcgctgtcgcccacaaccgcgccggtcaggactacggccgtgtcggtgtcggtgaggaaggcggccgaggggcagcggcggaccaccacctcggggcgcgggaccaccacctcggggcgcggggacgctgcggagcaggaggcgatgcgggaggcggtcctgtacgtgcgccgagagctctccgtcgtcgcgccggatgacctcacctcgccgcacaactag

SEQ ID NO: 27 - protein, 323 aa, Oryza sativa
MKGSDLPPGGSGQKPPGGPPGNRKGKRGAEAPPATSSSTPTTARRSKRLAGAPPDHPAEAGPSSTNARRSSRLAGAPPATPGAAAAAPTSSSPTAARRSNRLAGAPPDPPAAVAAPPTSSSPTTAMRSNRLAGAPSDPPAAATAGPTSPSPTTARRSNRLAGAPPDPPAAATAGPTSKTARRSKRLAGEAPETPVEAGQTSPSSTTARQSKRSAGKSPAIPKGSGQPSSAEKSKRTADASSADPAEARPSSLSPTTAPVRTTAVSVSVRKAAEGQRRTTTSGRGTTTSGRGDAAEQEAMREAVLYVRRELSVVAPDDLTSPHN

SEQ ID NO: 28 - DNA, 22 bp, Artificial Sequence
  Forward primer for cloning cDNA of OsHIS gene
acacacagctacaaatcgactg

SEQ ID NO: 29 - DNA, 22 bp, Artificial Sequence
  Reverse primer for cloning cDNA of OsHIS gene
gacacattgagcatttgatttg

SEQ ID NO: 30 - DNA, 37 bp, Artificial Sequence
  Forward primer for cloning gDNA of OsDN-FTG1gene
ctgctgaggacaatctctctatctctctctctcttcc

SEQ ID NO: 31 - DNA, 35 bp, Artificial Sequence
  Reverse primer for cloning gDNA of OsDN-FTG1 gene
gcgctgaggccaaggttaaacaagggtagagctaa

SEQ ID NO: 32 - DNA, 29 bp, Artificial Sequence
  Forward primer for cloning gDNA of OsWRKY76 gene
cctgtttctgttttgattactcgagctcc

SEQ ID NO: 33 - DNA, 30 bp, Artificial Sequence
  Reverse primer for cloning gDNA of OsWRKY76 gene
ctctaatctctattgaaatgcgcctctatg

SEQ ID NO: 34 - DNA, 28 bp, Artificial Sequence
  Forward primer for cloning cDNA of OsMYB77 gene
actcgtcgtggtagtagtagtcttcttc

SEQ ID NO: 35 - DNA, 30 bp, Artificial Sequence
  Reverse primer for cloning cDNA of OsMYB77 gene
gacgctaggtttttgatcagtgcatcctcc

SEQ ID NO: 36 - DNA, 24 bp, Artificial Sequence
  Forward primer for cloning gDNA of OsDN-FTG2 gene
gcagagattgagagatgttggtgc

SEQ ID NO: 37 - DNA, 22 bp, Artificial Sequence
  Reverse primer for cloning gDNA of OsDN-FTG2 gene
gtggaaagggggatcgatctcg

SEQ ID NO: 38 - DNA, 31 bp, Artificial Sequence
  Forward primer for cloning cDNA of OsENA1 gene
ctgctgaggcacccacccaccgatcatgttc

SEQ ID NO: 39 - DNA, 34 bp, Artificial Sequence
  Reverse primer for cloning cDNA of OsENA1 gene
ccgctgaggtgtaagaattcccagcacagagttg

SEQ ID NO: 40 - DNA, 34 bp, Artificial Sequence
  Forward primer for cloning cDNA of OsGRF1 gene
ctgctgagggtccaggactccgtacaattcagca

SEQ ID NO: 41 - DNA, 34 bp, Artificial Sequence
  Reverse primer for cloning cDNA of OsGRF1 gene
ccgctgagggagtgtgtgtgtgtgtgagggagag

SEQ ID NO: 42 - DNA, 27 bp, Artificial Sequence
  Forward primer for cloning cDNA of OsHIP14 gene
cagacatcaattgataaagagcacatc

SEQ ID NO: 43 - DNA, 28 bp, Artificial Sequence
  Reverse primer for cloning cDNA of OsHIP14 gene
gtgtttactgccctgtgctttttctaag

SEQ ID NO: 44 - DNA, 33 bp, Artificial Sequence
  Forward primer for cloning gDNA of OsDN-FTG3 gene
ctgctgaggagcaaggaaatacccaaccccgag

SEQ ID NO: 45 - DNA, 29 bp, Artificial Sequence
  Reverse primer for cloning gDNA of OsDN-FTG3 gene
ccgctgaggccaccctcggctagttgtgc

SEQ ID NO: 46 - DNA, 18 bp, Artificial Sequence
  Forward primer for real-time PCR analysis of OsHIS gene
actactacccgccgatgg

SEQ ID NO: 47 - DNA, 19 bp, Artificial Sequence
  Reverse primer for real-time PCR analysis of OsHIS gene
caaaaccttgctgcccttc

SEQ ID NO: 48 - DNA, 20 bp, Artificial Sequence
  Forward primer for real-time PCR analysis of OsDN-FTG1 gene
ggagattgttgctggggatg

SEQ ID NO: 49 - DNA, 18 bp, Artificial Sequence
  Reverse primer for real-time PCR analysis of OsDN-FTG1 gene
gaacccgatggcatcctc

SEQ ID NO: 50 - DNA, 20 bp, Artificial Sequence
  Forward primer for real-time PCR analysis of OsWRKY76 gene
ccatcacgctcgacctcacc

SEQ ID NO: 51 - DNA, 20 bp, Artificial Sequence
  Reverse primer for real-time PCR analysis of OsWRKY76 gene
ggtgaacttggggtcgctgg

SEQ ID NO: 52 - DNA, 20 bp, Artificial Sequence
  Forward primer for real-time PCR analysis of OsMYB77 gene
acagccacttccaagaactc

SEQ ID NO: 53 - DNA, 19 bp, Artificial Sequence
  Reverse primer for real-time PCR analysis of OsMYB77 gene
atgtacttgtgcacctcgg

SEQ ID NO: 54 - DNA, 20 bp, Artificial Sequence
  Forward primer for real-time PCR analysis of OsDN-FTG2 gene
tcaagtacatcgacaaggcg

SEQ ID NO: 55 - DNA, 18 bp, Artificial Sequence
  Reverse primer for real-time PCR analysis of OsDN-FTG2 gene
gtggatggcttgacggag

SEQ ID NO: 56 - DNA, 19 bp, Artificial Sequence
  Forward primer for real-time PCR analysis of OsENA1 gene
ggagttcgaccacttcgtg

SEQ ID NO: 57 - DNA, 19 bp, Artificial Sequence
  Reverse primer for real-time PCR analysis of OsENA1 gene
atgatctcctgcgggtaga

SEQ ID NO: 58 - DNA, 20 bp, Artificial Sequence
  Forward primer for real-time PCR analysis of OsDN-FTG3 gene
cgcagcgctggtaagtctcc

SEQ ID NO: 59 - DNA, 19 bp, Artificial Sequence
  Reverse primer for real-time PCR analysis of OsDN-FTG3 gene
cacggccgtagtcctgacc

SEQ ID NO: 60 - DNA, 741 bp, Oryza sativa; identical to SEQ ID NO: 2

SEQ ID NO: 61 - protein, 246 aa, Oryza sativa; identical to SEQ ID NO: 3

SEQ ID NO: 62 - DNA, 738 bp, Zea mays
atggacaaccagccgctgccctactccacaggccagccccctgcccccggaggagccccggtggcgggcatgcctggcgcggccggcctcccacccgtgccgcaccaccacctgctccagcagcagcaggcccagctgcaggcgttctgggcgtaccagcgccaggaggcggagcgcgcgtccgcgtcggacttcaagaaccaccagctgcctctggcccggatcaagaagatcatgaaggccgacgaggacgtgcgcatgatctccgccgaggcgcccgtgctgttcgccaaggcctgcgagctcttcatcctcgagctcactatccgctcctggctccacgccgaggagaacaagcgccgcaccctgcagcgcaacgacgtcgccgcggccatcgcgcgcaccgacgtcttcgatttcctcgtcgacatcgtgccccgcgaggaggccaaggaggagcccggcagcgccctcggcttcgcggcgcctgggaccggcgtcgtcggggctggcgccccgggcggggcgccagccgccgggatgccctactactatccgccgatggggcagccggcgccgatgatgccggcctggcatgttccggcctgggacccggcctggcagcaaggggcagcggatgtcgatcagagcggcagcttcagcgaggaaggacaagggtttggagcaggccatggcggcgccgctagcttccctcctgcgcctccgacctccgagtga

SEQ ID NO: 63 - protein, 245 aa, Zea mays
MDNQPLPYSTGQPPAPGGAPVAGMPGAAGLPPVPHHHLLQQQQAQLQAFWAYQRQEAERASASDFKNHQLPLARIKKIMKADEDVRMISAEAPVLFAKACELFILELTIRSWLHAEENKRRTLQRNDVAAAIARTDVFDFLVDIVPREEAKEEPGSALGFAAPGTGVVGAGAPGGAPAAGMPYYYPPMGQPAPMMPAWHVPAWDPAWQQGAADVDQSGSFSEEGQGFGAGHGGAASFPPAPPTSE

SEQ ID NO: 64 - DNA, 744 bp, Sorghum bicolor
atggacaaccagccgctgccctactccaccggccagccccctgcccccggaggaaccccagtagtgccgggcgtgcccggcgcagccggccctccgccggtgccgcaccaccacctgctccagcagcagcaggcccagctgcaggcgttctgggcgtaccagcgccaggaggcggagcgcgcgtcggcgtcggacttcaagaaccaccagctgccgctggcccggatcaagaagatcatgaaggccgacgaggacgtgcgcatgatctccgccgaggcgcccgtgctgttcgccaaggcctgcgagctcttcatcctcgagctcaccatccggtcctggctgcacgccgaggagaacaagcgccgcaccctgcagcggaacgacgtcgccgcggccatcgcgcgcaccgacgtcttcgacttcctcgtcgacatcgtgccccgcgaggaggccaaggaggagcccggcagcgccctcggcttcgcggcgtccgggaccggcgtcgtcgggggtggcgccccgggcggggcgccggccgccgggatgccgtactactatccgccgatggggcagccggcgccgatgatgccggcctggcatgttccggcctgggacccggcctggcagcaaggggcagccgatgtcgatcacagcggcagcttcagcgaggaaggacaagcagggtttgctgcaggccatggcggccccgctagcttccctcctgcgcctccgagctccgagtga

SEQ ID NO: 65 - protein, 247 aa, Sorghum bicolor
MDNQPLPYSTGQPPAPGGTPVVPGVPGAAGPPPVPHHHLLQQQQAQLQAFWAYQRQEAERASASDFKNHQLPLARIKKIMKADEDVRMISAEAPVLFAKACELFILELTIRSWLHAEENKRRTLQRNDVAAAIARTDVFDFLVDIVPREEAKEEPGSALGFAASGTGVVGGGAPGGAPAAGMPYYYPPMGQPAPMMPAWHVPAWDPAWQQGAADVDHSGSFSEEGQAGFAAGHGGPASFPPAPPSSE

SEQ ID NO: 66 - DNA, 753 bp, Arabidopsis thaliana
atggacaataacaacaacaacaacaaccagcaaccaccaccaacctccgtctatccacctggctccgccgtcacaaccgtaatccctcctccaccatctggatctgcatcaatagtcaccggaggaggagcgacataccaccacctcctccagcaacaacagcaacagcttcaaatgttctggacataccagagacaagagatcgaacaggtaaacgatttcaaaaaccatcagctccctctagctcgtatcaaaaaaatcatgaaagctgatgaagatgtgcgtatgatctccgccgaagcaccgattctcttcgcgaaagcttgtgagcttttcattctcgaacttacgattagatcttggcttcacgctgaagagaacaaacgtcgtacgcttcagaaaaacgatatcgctgctgcgattactagaaccgatatcttcgatttccttgttgatattgttcctagggaagagatcaaggaagaggaagatgcagcatcggctcttggtggaggaggtatggttgctcccgccgcgagcggtgttccttattattatccaccgatgggacaaccggcggttcctggagggatgatgattggaagaccggcgatggatcctagcggtgtttatgctcagcctccttctcaggcatggcaaagcgtttggcagaattcagctggtggtggtgatgatgtgtcttatggaagtggaggaagtagcggccatggtaatctcgatagccaaggttga

SEQ ID NO: 67 - protein, 250 aa, Arabidopsis thaliana
MDNNNNNNNQQPPPTSVYPPGSAVTTVIPPPPSGSASIVTGGGATYHHLLQQQQQQLQMFWTYQRQEIEQVNDFKNHQLPLARIKKIMKADEDVRMISAEAPILFAKACELFILELTIRSWLHAEENKRRTLQKNDIAAAITRTDIFDFLVDIVPREEIKEEEDAASALGGGGMVAPAASGVPYYYPPMGQPAVPGGMMIGRPAMDPSGVYAQPPSQAWQSVWQNSAGGGDDVSYGSGGSSGHGNLDSQG

SEQ ID NO: 68 - DNA, 678 bp, Glycine max
atggagaacaaccagcaacaaggcgctcaagcccaatcgggaccgtaccccggcggcgccggtggaagtgcaggtgcaggtgcaggtgcaggcgcggccccgttccagcacctgctccagcagcagcagcagcagctgcagatgttctggtcgtaccagcggcaagagatcgagcacgtgaacgacttcaagaaccaccagctccccttggcccgcatcaagaagatcatgaaggccgacgaggacgtccgcatgatctccgccgaggcccccatcctcttcgccaaggcctgcgagctcttcatcctcgagctcaccatccgctcctggctccacgccgacgagaacaagcgccgcaccctccagaagaacgacatcgccgccgccatcactcgcaccgacattttcgacttcctcgtcgacatcgtcccccgcgacgagatcaaggacgacgccgcgctcgtcggggcaacggccagtggggtgccttactactacccgcccattggccagcctgccgggatgatgattggccgccccgccgtcgatcccgccaccggagtttatgtccagccgccctcccaggcctggcagtccgtctggcagtccgccgccgaggacacgccctacggcaccggtgcccaggggaaccttgatggccagagtatggaattctag

SEQ ID NO: 69 - protein, 225 aa, Glycine max
MENNQQQGAQAQSGPYPGGAGGSAGAGAGAGAAPFQHLLQQQQQQLQMFWSYQRQEIEHVNDFKNHQLPLARIKKIMKADEDVRMISAEAPILFAKACELFILELTIRSWLHADENKRRTLQKNDIAAAITRTDIFDFLVDIVPRDEIKDDAALVGATASGVPYYYPPIGQPAGMMIGRPAVDPATGVYVQPPSQAWQSVWQSAAEDTPYGTGAQGNLDGQSMEF

SEQ ID NO: 70 - DNA, 402 bp, Zea mays
atgctcgcgacccgcggtgaaatcctggagccgaacctcggcgtcggtggcggaggaggacgggcctgcgaaggacagcatggtcgagccagggtccgtcgccgccgcgccccacgacggagacccggaagccgctgcccagtccatgccgccgctggccaagaaggaaaggagcacctccgccctccaagccccagatctggccgtcgagcgcgggctccggtgcccccgcgcgcccagcctcctcctccgaagaggaggcggcgcccgtcggcggcaaccccagctcatcgtccgaggcctcgagtaggaagcccagctctggctgctgccccggtgccggcgccggcgcctccacggcctcgtcaaagctggctatctcctcctacaggctccgcatga

SEQ ID NO: 71 - protein, 133 aa, Zea mays
MLATRGEILEPNLGVGGGGGRACEGQHGRARVRRRRAPRRRPGSRCPVHAAAGQEGKEHLRPPSPRSGRRARAPVPPRAQPPPPKRRRRPSAATPAHRPRPRVGSPALAAAPVPAPAPPRPRQSWLSPPTGSA

SEQ ID NO: 72 - DNA, 609 bp, Sorghum bicolor
atggagagctcttctcacaagcgggcgcgggaggtggtggacctcaccgccgccgggcccggagaggcagccgcgttgccggaggcggacgcgaagaggctgcggccgcaggatctcctggacatgctcgatgacgacaccgacgcggccgccgccggcgacctagcgtccgtcatgaggagcctggaggaggagatagctagcttcgacgaggccgtgggggccgcggaagcggcgcctacgcagcagcagcagcagccggagctggggttcctgctggaggcctcggacgacgagctggggctgccaccggctggcgcctccgcctcgtcgtcggaggaggccggcggcggcggcgggggcctcgccggggcgccgccggagtccgccgagctggacggcggccagatctggggcttcgaggacgagatagacggtggagcgggcttcggtggctactcgccggaggcggccgccgcggccgtcgccgccgcggcggcgtgggacgacgacggcttcgacgccggcctgttcgcgttcggcgaaagcgacgcgtacgggccgtcggatctcgccgcgctgcgccacgagaccatgccggccgtctga

SEQ ID NO: 73 - protein, 202 aa, Sorghum bicolor
MESSSHKRAREVVDLTAAGPGEAAALPEADAKRLRPQDLLDMLDDDTDAAAAGDLASVMRSLEEEIASFDEAVGAAEAAPTQQQQQPELGFLLEASDDELGLPPAGASASSSEEAGGGGGGLAGAPPESAELDGGQIWGFEDEIDGGAGFGGYSPEAAAAAVAAAAAWDDDGFDAGLFAFGESDAYGPSDLAALRHETMPAV

SEQ ID NO: 74 - DNA, 585 bp, Arabidopsis thaliana
atggagaagaagttgttggatataactcggactgactcggctgagaaaaagcgagtcagagacgagtcattcgacgaagcggttcttgactcgccggaggtgaagaggttgagagatgatttattcgatgtcctggatgactcggatcctgaaccagtgagtcaagatctcgactcggttatgaaaagtttcgaagacgagttatcaacggtcaccaccacgacggcgcaaggttcctctaccgccggcgaaactcagcctgatctcggatatcttcttgaagcttccgatgatgagcttggtttaccaccacctccatcgatttctccggttcccgtcgcgaaggaggaggtaacgacggagacggtaacggatttggtacgagcgtcttctgattcgtcaggaatcgacgagatttggggatttgaagatcacgtgtcgaattacggtggtttagattttggttccggcgtcggagatggtggagattacgtggctgttgaggggttgtttgaattttccgacgattgttttgactccggcgatctgttttcgtggcggtcggagtcgttaccggcggaataa

SEQ ID NO: 75 - protein, 194 aa, Arabidopsis thaliana
MEKKLLDITRTDSAEKKRVRDESFDEAVLDSPEVKRLRDDLFDVLDDSDPEPVSQDLDSVMKSFEDELSTVTTTTAQGSSTAGETQPDLGYLLEASDDELGLPPPPSISPVPVAKEEVTTETVTDLVRASSDSSGIDEIWGFEDHVSNYGGLDFGSGVGDGGDYVAVEGLFEFSDDCFDSGDLFSWRSESLPAE

SEQ ID NO: 76 - DNA, 1047 bp, Oryza sativa
atggatccgtggattagcacccagccttcgctgagcctggacctccgcgtcgggctgccggcgacggcggccgtcgccatggttaagcccaaggtgctcgtcgaggaggacttctttcaccagcagcctctcaagaaagacccagaggttgcggcgctggaggcggagctgaagcggatgggcgcggagaaccggcagctgagcgagatgctggcggcggtggcggccaagtacgaggcgctgcagagccagttcagcgacatggtcaccgccagcgccaacaacggcggcggcggcggcaacaacccgtcgtccacctccgagggcggctccgtctcgccgtcgaggaagcgcaagagcgagagcctcgacgactccccgccgccgccgccgccgccgcacccacacgcggcgccgcaccacatgcacgtcatgcccggcgccgccgccgccggctacgccgaccagaccgagtgcacctccggcgagccctgcaagcgcatccgcgaggagtgcaagcccaagatctccaagctctacgtccacgccgacccatccgacctcagcctggtggtgaaagatgggtaccaatggaggaagtatggtcagaaggtcaccaaggacaacccctgcccaagagcctacttcagatgctcatttgctcccgcctgccctgtcaagaagaaggttcagagaagcgcggaggacaacacgatcctcgtggcgacgtacgagggggagcacaaccacggccagccgccgccgccgctgcagtcggcggcgcagaacagcgacggctccggcaagagcgccgggaagccaccccatgcgccggcggcggcgccgccggcgccggtggtgccgcaccgtcagcacgaaccggtcgtcgtcaacggcgagcagcaggccgcggcggcgtcggagatgatcaggcggaacctggccgagcagatggcgatgacgctgacgcgtgacccaagcttcaaggcggcgctcgtcaccgccctctccggccgcatcctcgagctctcgccgaccaaggattga

SEQ ID NO: 77 - protein, 348 aa, Oryza sativa
MDPWISTQPSLSLDLRVGLPATAAVAMVKPKVLVEEDFFHQQPLKKDPEVAALEAELKRMGAENRQLSEMLAAVAAKYEALQSQFSDMVTASANNGGGGGNNPSSTSEGGSVSPSRKRKSESLDDSPPPPPPPHPHAAPHHMHVMPGAAAAGYADQTECTSGEPCKRIREECKPKISKLYVHADPSDLSLVVKDGYQWRKYGQKVTKDNPCPRAYFRCSFAPACPVKKKVQRSAEDNTILVATYEGEHNHGQPPPPLQSAAQNSDGSGKSAGKPPHAPAAAPPAPVVPHRQHEPVVVNGEQQAAAASEMIRRNLAEQMAMTLTRDPSFKAALVTALSGRILELSPTKD

SEQ ID NO: 78 - DNA, 909 bp, Zea mays
atgatgctgctcatggactcggggagccgcggtgactgctccccggtctgcttggacctcagcgtcggcctttcgccgccgtcgccggggagcggcccggaaacgacagctgacgctgagagactcgaccgtcccgccactggctgctgggggccacggacatcgtccctcgctgacgggaaggacgaggccaagaccctggaggccaggctcacccaggtcagcgaggagaaccggcgtctcaccgagatcatcgcctacatgtacgccagccaggtcgccgcgcggcggagtcccgacggcaggaagaggagcagggacagcctggagccgtcgaattcgggcgacgccaacgctgccgtcgagagcgccgccctcagcgacgagggcacgtgcaggcggatcaagctcaccagggtctgcaccaagatcgacccctccgacaccacgctcaccgtcaaagacggctaccagtggcgcaagtacggccagaaggtgacgcgcgacaacccgtccccgagagcctacttccgctgcgcatacgcaccatcctgccccgtcaagaagaaggtgcagaggagcgcggaggacagcgccatgctggtggccacgtacgagggcgagcacaaccacccgagcccgacgcgcgccggcgagctccccagctccacctccatcaactcctccggcccggccatcacgctggacctcaccaggaacggagccggcgccgtgcgggggctcgatgccgccgccgaggtgcccggcctcaagaggctgtgccaggagatcgcgtcaccggatttccgcacggcgctcgtggagcagatggcgcgctcgctcaccaaggatcccaagttcaccgacgcgctggctgccgcgatcctgcagcagctgccggagtactag

SEQ ID NO: 79 - protein, 302 aa, Zea mays
MMLLMDSGSRGDCSPVCLDLSVGLSPPSPGSGPETTADAERLDRPATGCWGPRTSSLADGKDEAKTLEARLTQVSEENRRLTEIIAYMYASQVAARRSPDGRKRSRDSLEPSNSGDANAAVESAALSDEGTCRRIKLTRVCTKIDPSDTTLTVKDGYQWRKYGQKVTRDNPSPRAYFRCAYAPSCPVKKKVQRSAEDSAMLVATYEGEHNHPSPTRAGELPSSTSINSSGPAITLDLTRNGAGAVRGLDAAAEVPGLKRLCQEIASPDFRTALVEQMARSLTKDPKFTDALAAAILQQLPEY

SEQ ID NO: 80 - DNA, 984 bp, Sorghum bicolor
atgctgctcatggactcggcgcgccgcgccggctgctccccgtccccggtctgcttggacctcagcgtcggcctttcgccgtcgtcgccggggagcagcggcccggaaacgacagctgacactgacgacaggcttgaccgtcccgccgctggctgcagggtggcatcgtccctgtctgacgagcaggccaagaccctggaggccaagctcacccaggtcagcgaggagaaccgccggctcaccgagatgatcgcctacctgtacgccagccaggtcgcgcggcagagctccagctcccccgacaccaccagcaggaagaggagcagggacagcctggagccgccgtcgaattccagcgacggcaacgccaacgccaaggcggagcccggcgaccatgccgccgtcgagagcgccctcagcgacgagggcacgtgcaggcggatcaaggtcaccagggtctgcacccggatcgaccccgccgacgccacgctcaccgtcaaagacggctaccaatggcgaaagtacggccagaaggtgacccgcgacaacccgtccccgagagcctacttccgctgcgcatacgctccctcctgccccgtcaagaagaaggtgcagaggagcgcggaggacagctccttgctggtggcgacgtacgagggcgagcacaaccacccgagcccgacgcgcgccggcgagctccccagctccgcctccgcgacggccagcggccccgtgccgtgctccatctccatcaactcctccggcccgaccatcacgctggacctcaccaagaacggagggggaggcggcgtgcgggtgctcgacgccgccgaggcgcccgacctcaagaagctgtgccaggagatcgcgtcgccggatttccggacggcgctcgtggagcagatggcgcgctcgctgaccagcgattccaagttcacccacgcgctggctgccgcgatcctgcagcagctgccggagtactag

SEQ ID NO: 81 - protein, 327 aa, Sorghum bicolor
MLLMDSARRAGCSPSPVCLDLSVGLSPSSPGSSGPETTADTDDRLDRPAAGCRVASSLSDEQAKTLEAKLTQVSEENRRLTEMIAYLYASQVARQSSSSPDTTSRKRSRDSLEPPSNSSDGNANAKAEPGDHAAVESALSDEGTCRRIKVTRVCTRIDPADATLTVKDGYQWRKYGQKVTRDNPSPRAYFRCAYAPSCPVKKKVQRSAEDSSLLVATYEGEHNHPSPTRAGELPSSASATASGPVPCSISINSSGPTITLDLTKNGGGGGVRVLDAAEAPDLKKLCQEIASPDFRTALVEQMARSLTSDSKFTHALAAAILQQLPEY

SEQ ID NO: 82 - DNA, 909 bp, Arabidopsis thaliana
atggatcagtactcatcctctttggtcgatacttcattagatctcactattggcgttactcgtatgcgagttgaagaagatccaccgacaagtgctttggtggaagaattaaaccgagttagtgctgagaacaagaagctctcggagatgctaactttgatgtgtgacaactacaacgtcttgaggaagcaacttatggaatatgttaacaagagcaacataaccgagagggatcaaatcagccctcccaagaaacgcaaatccccggcgagagaggacgcattcagctgcgcggttattggcggagtgtcggagagtagctcaacggatcaagatgagtatttgtgtaagaagcagagagaagagactgtcgtgaaggagaaagtctcaagggtctattacaagaccgaagcttctgacactaccctcgttgtgaaagatgggtatcaatggaggaaatatggacagaaagtgactagagacaatccatctccaagagcttacttcaaatgtgcttgtgctccaagctgttctgtcaaaaagaaggttcagagaagtgtggaggatcagtccgtgttagttgcaacttatgagggtgaacacaaccatccaatgccatcgcagatcgattcaaacaatggcttaaaccgccacatctctcatggtggttcagcttcaacacccgttgcagcaaacagaagaagtagcttgactgtgccggtgactaccgtagatatgattgaatcgaagaaagtgacgagcccaacgtcaagaatcgattttccccaagttcagaaacttttggtggagcaaatggcttcttccttaaccaaagatcctaactttacagcagctttagcagcagctgttaccggaaaattgtatcaacagaatcataccgagaaatag

SEQ ID NO: 83 - protein, 302 aa, Arabidopsis thaliana
MDQYSSSLVDTSLDLTIGVTRMRVEEDPPTSALVEELNRVSAENKKLSEMLTLMCDNYNVLRKQLMEYVNKSNITERDQISPPKKRKSPAREDAFSCAVIGGVSESSSTDQDEYLCKKQREETVVKEKVSRVYYKTEASDTTLVVKDGYQWRKYGQKVTRDNPSPRAYFKCACAPSCSVKKKVQRSVEDQSVLVATYEGEHNHPMPSQIDSNNGLNRHISHGGSASTPVAANRRSSLTVPVTTVDMIESKKVTSPTSRIDFPQVQKLLVEQMASSLTKDPNFTAALAAAVTGKLYQQNHTEK

SEQ ID NO: 84 - DNA, 942 bp, Glycine max
atggattgttcatcatggattaacacttccttggatctcagcattaatcctcgcagagttcatgaagaagctgttcctaaggtggtagaaagcaagcttttctctttgggaatgcccaagtttaacgtcgaagaagagtctactagtgacttggaggaggaactgaagcgggtgagtgcagaaaacaagaagttggccgaaatgctctcagtggtgtgtgagaattacaacactttgagaagccatttgatggaatacatgaggaaaaatggcgaaaaggaggtcagcccaacatcaaagaaaagaaagtctgaaagcagcaacaacaacaacagtaatttgatgggaactaacaatggaaactcagagagcagttctactgatgaagagtcttgcaagaaaccaagggaggaaaccatcaaagcaaaaatttcaagagtttatgtcaggactgaatcatctgatactagccttattgtgaaagatggataccaatggaggaaatatggacaaaaggtgaccagagataacccttaccctagagcatatttcaagtgctcttttgctccaagctgccctgtcaaaaagaaggtgcaaagaagtgtggatgatcattctgttctgcttgctacttatgaaggggagcacaatcatcctcaggcttcttcccaaatggaagcaacatcaggttctggccgtagtgtgacccttggttcagtgccttgttcagcatctctcagcacttccactccaacacttgttacccttgacttgacaaaatctaagggaagcaacgattccaagagcacaaaacctaaaggagattcacctaaagtacctcaggttttggtggaacagatggctacttctttgaccacggatcctaattttagagcagcacttgttgctgccatctcaggaagattgttgcacaataattaa

SEQ ID NO: 85 - protein, 313 aa, Glycine max
MDCSSWINTSLDLSINPRRVHEEAVPKVVESKLFSLGMPKFNVEEESTSDLEEELKRVSAENKKLAEMLSVVCENYNTLRSHLMEYMRKNGEKEVSPTSKKRKSESSNNNNSNLMGTNNGNSESSSTDEESCKKPREETIKAKISRVYVRTESSDTSLIVKDGYQWRKYGQKVTRDNPYPRAYFKCSFAPSCPVKKKVQRSVDDHSVLLATYEGEHNHPQASSQMEATSGSGRSVTLGSVPCSASLSTSTPTLVTLDLTKSKGSNDSKSTKPKGDSPKVPQVLVEQMATSLTTDPNFRAALVAAISGRLLHNN

SEQ ID NO: 86 - DNA, 906 bp, Oryza sativa
atggggatggaggcggagtgcgataggataaaggggccatggagccctgaggaggacgaggcgctgcggcggctggtggagcggcacggggcgaggaactggacggcgatcgggagggggatcccggggaggtcggggaagtcgtgcaggctgcggtggtgcaaccagctgtcgccgcaggtggagcgccgcccgttcacggcggaggaggacgccgcgatactccgggcgcacgcccgcctcggcaaccgctgggccgccatcgcgcgcctcctccccggccgcaccgacaacgccgtcaagaaccactggaactcctccctcaagcgcaagctcgccaccgccacggacggcggcgagatcgaccggccgtgtaagcgcgtgagccccgggccggggagcccgacgggatcggagcgcagcgagctcagccacggcggctgcggaagcggaagcggcggcgggcaggtgttccgccccgtgccgcgccccggcggcttcgacgccatcagcgcggcggacgtcgtgcggccgccgcggcggcgggacgacaacgacgacgacggcgacgacgacccgctcacctcgctgtccctctccctctccctcccggggttccaccacgacagcgcgcggagccacttccaggagctcccctccccctcccgctccccttcgccgccgccgtcgccgccggcagcatctccatccgcatacccgttcaacgccgatctcgtctccgcgatgcaggagatgatccgcacggaagtccgcaactacatggccggcgtcgggctccgcgccggctgcggcccgggcgccgtggccgagtccttcatgccgcagctcgtcgacggcgtcatgcgcgccgccgccgagagggtcggcgtcgtgacccgccaataa

SEQ ID NO: 87 - protein, 301 aa, Oryza sativa
MGMEAECDRIKGPWSPEEDEALRRLVERHGARNWTAIGRGIPGRSGKSCRLRWCNQLSPQVERRPFTAEEDAAILRAHARLGNRWAAIARLLPGRTDNAVKNHWNSSLKRKLATATDGGEIDRPCKRVSPGPGSPTGSERSELSHGGCGSGSGGGQVFRPVPRPGGFDAISAADVVRPPRRRDDNDDDGDDDPLTSLSLSLSLPGFHHDSARSHFQELPSPSRSPSPPPSPPAASPSAYPFNADLVSAMQEMIRTEVRNYMAGVGLRAGCGPGAVAESFMPQLVDGVMRAAAERVGVVTRQ

SEQ ID NO: 88 - DNA, 915 bp, Zea mays
atgggctcggaggcggactgcgacaggatcaggggcccgtggagccccgaggaggacgacgcgctgcggcggctcgtggagcggcacggcgcgcgcaactggacggcgatcgggcgcgagatcccggggcggtcgggcaagtcgtgccgcctgcggtggtgcaaccagctgtcgccgcaggtggagcgccggcccttcacggcggaggaggacgcggccatcgtccgcgcccacgcccgcctcggcaaccgctgggccgccatcgcgcgcctcctccccggccgcaccgacaacgccgtcaagaaccactggaactgctcgctcaagcgcaagctcgccgccgcgtccgccccggcgggttcctccgacgtcgaggcgcgcccgaccaagcgcgtcagcctctcgcccgacagcccgtcggggtcggggtcggggtcagggtcagggtccgggtccgggtccgggtccgaccgtagcgacctgagccacggcgccggccaggtttaccgcccagtggcgcggtccggcgggttcgagccggcggactgcgcgatgagccgtccgctggaggaagaggaggaggaaaaggaggacccgctcacctccctcagcctctcgcttccaggcacggaccagcggttccaccacgaccgcgcccacagccagttccaggagctcccggcgtcgccgccctcgccctccccgcctccgccgccggttctctcggcgtacccgttcagccccgtgttcatggaggcgatgcaggagctgatccgcacggaggtgcagcggtacatggcgagcgtgggcgtgcgcgccgggtgcggcgccgccggcggggccgacctctgcatgccgcaactggtcgacggcgtcatgcgcgccgccgccgagcgggtcggccggatgcagtag

SEQ ID NO: 89 - protein, 304 aa, Zea mays
MGSEADCDRIRGPWSPEEDDALRRLVERHGARNWTAIGREIPGRSGKSCRLRWCNQLSPQVERRPFTAEEDAAIVRAHARLGNRWAAIARLLPGRTDNAVKNHWNCSLKRKLAAASAPAGSSDVEARPTKRVSLSPDSPSGSGSGSGSGSGSGSGSDRSDLSHGAGQVYRPVARSGGFEPADCAMSRPLEEEEEEKEDPLTSLSLSLPGTDQRFHHDRAHSQFQELPASPPSPSPPPPPVLSAYPFSPVFMEAMQELIRTEVQRYMASVGVRAGCGAAGGADLCMPQLVDGVMRAAAERVGRMQ

SEQ ID NO: 90 - DNA, 942 bp, Sorghum bicolor
atgggcgcggaggcggagtgcgacaggatcagggggccgtggagccccgaggaggacgacgcgctgcggcggctggtggagcggcacggcgcgcgcaactggacggcgatcgggcgcgagatccctggccggtcgggcaagtcgtgccgcctgcggtggtgcaaccagctgtcgccgcaggtggagcgccggcccttcacgcccgaggaggacgccgccatcgtccgcgcccacgcgcggctcggcaaccgctgggccgccatcgcgcgcctcctcccagggcgcaccgacaacgccgtcaagaaccactggaactgctccctcaagcgcaagctcgccgtcgccaccaccgccgccgccgtgtccggttcgggagtggtctccgctgacgccgccgccgagatcgaggcgacgcgcccgatcaagcgcgtcagcctctcgcccgacagcccgtcgggttccgggtcggggtccgggtctaggtccgaccgtagcgacctcagccacggctccggctccggctccggccagatttaccgcccggtggcgcggtccggtgggttcgagccagcagactgcgcgatgagccgtccgcaggaggacgacgacccgctgacctccctcagcctgtcgctcccgggcacggaccaccagcggttccaccacgaccgcgcccacagccagttccaggagctcccggcgtcgccgccctcaccctccccgccttctccggcggcggcaccaccctcggcgtacccgttcagcccggacttcatggcggcgatgcaggagctgatccgcacggaggtgcagcggtacatggcgagcgtgggcgtgcgcgccgggtgcggcgccacgggcggtgccgccgacctctgcatgccgcagcttgtcgagggcgtcatgcgcgccgccgccgagcgagtcggccggatgcagtag

SEQ ID NO: 91 - protein, 313 aa, Sorghum bicolor
MGAEAECDRIRGPWSPEEDDALRRLVERHGARNWTAIGREIPGRSGKSCRLRWCNQLSPQVERRPFTPEEDAAIVRAHARLGNRWAAIARLLPGRTDNAVKNHWNCSLKRKLAVATTAAAVSGSGVVSADAAAEIEATRPIKRVSLSPDSPSGSGSGSGSRSDRSDLSHGSGSGSGQIYRPVARSGGFEPADCAMSRPQEDDDPLTSLSLSLPGTDHQRFHHDRAHSQFQELPASPPSPSPPSPAAAPPSAYPFSPDFMAAMQELIRTEVQRYMASVGVRAGCGATGGAADLCMPQLVEGVMRAAAERVGRMQ

SEQ ID NO: 92 - DNA, 918 bp, Arabidopsis thaliana
atggctgataggatcaaaggtccatggagtcctgaagaagacgagcagcttcgtaggcttgttgttaaatacggtccaagaaactggacagtgattagcaaatctattcccggtagatcggggaaatcgtgtcgtttacggtggtgcaaccagctttcgccgcaagttgagcatcggccgttttcggctgaggaagacgagacgatcgcacgtgctcacgctcagttcggtaataaatgggcgacgattgctcgtcttctcaacggtcgtacggacaacgccgtgaagaatcactggaactcgacgctcaagaggaaatgcggcggttacgaccatcggggttacgatggttcggaggatcatcggccggttaagagatcggtgagtgcgggatctccacctgttgttactgggctttacatgagcccaggaagcccaactggatctgatgtcagtgattcaagtactatcccgatattaccttccgttgagcttttcaagcctgtgcctagacctggtgctgttgtgctaccgcttcctatcgaaacgtcgtcttcttccgatgatccaccgacttcgttaagcttgtcacttcctggtgccgacgtaagcgaggagtcaaaccgtagccacgagtcaacgaatatcaacaacaccacttcgagccgccacaaccacaacaatacggtgtcgtttatgccgtttagtggtgggtttagaggtgcgattgaggaaatggggaagtcttttcccggtaacggaggcgagtttatggcggtggtgcaagagatgattaaggcggaagtgaggagttacatgacggagatgcaacggaacaatggtggcggattcgtcggaggattcattgataatggcatgattccgatgagtcaaattggagttgggagaatcgagtag

SEQ ID NO: 93 - protein, 305 aa, Arabidopsis thaliana
MADRIKGPWSPEEDEQLRRLVVKYGPRNWTVISKSIPGRSGKSCRLRWCNQLSPQVEHRPFSAEEDETIARAHAQFGNKWATIARLLNGRTDNAVKNHWNSTLKRKCGGYDHRGYDGSEDHRPVKRSVSAGSPPVVTGLYMSPGSPTGSDVSDSSTIPILPSVELFKPVPRPGAVVLPLPIETSSSSDDPPTSLSLSLPGADVSEESNRSHESTNINNTTSSRHNHNNTVSFMPFSGGFRGAIEEMGKSFPGNGGEFMAVVQEMIKAEVRSYMTEMQRNNGGGFVGGFIDNGMIPMSQIGVGRIE

SEQ ID NO: 94 - DNA, 888 bp, Glycine max
atgatagccgtggcccgaaaggacatggaccggatcaagggcccgtggagcccggaggaggacgaggcccttcagaagcttgtggagcggcacgggcccagaaactggtcgctcatcagcaggtccattccgggccggtctgggaagtcatgcaggcttcggtggtgcaaccagctgtcgccccaggtcgagcaccgggccttcacaccggaagaggacgagaccattattcgggcccatgcccggtttggtaacaagtgggccaccattgcgcgcctcctctcgggccgcaccgataacgccatcaagaatcactggaactcaaccctaaaacgcaagtgcgcgtccttcatgatggcgggtgatgaagccgtcgccgtgagtccgaggccgctcaagcgatccttcagcgccggcgcggcggtgcctcctcccggaagcccctccggatccgatttcagcgagtccagcgctcccggcgtggtctcggtctctccttcgcacgtgtttcgccccgtgccggttcggccgattgttgaaacggcgtcgtcgcaagacgatgccgacgatgccggacccgcgacttcgctgtcgctgtctcttcccggtgtggagtcggcggaaatttcaaatcgcgcaactacggtgccggtgatgccggtgaataccgttgcggctccggcaccggttccggcggaggttggattgggagcgttgaatttgagtggagagtttatggcggtgatgcatgagatgataaggaaggaggtgaggagttacatggagcagcagaagaatgggatgatgtgttttcagggtatggaaatgatggaagggtttaggaacgtgtcggtgaagcgaattgggattagcagggtggattcgtaa

SEQ ID NO: 95 - protein, 295 aa, Glycine max
MIAVARKDMDRIKGPWSPEEDEALQKLVERHGPRNWSLISRSIPGRSGKSCRLRWCNQLSPQVEHRAFTPEEDETIIRAHARFGNKWATIARLLSGRTDNAIKNHWNSTLKRKCASFMMAGDEAVAVSPRPLKRSFSAGAAVPPPGSPSGSDFSESSAPGVVSVSPSHVFRPVPVRPIVETASSQDDADDAGPATSLSLSLPGVESAEISNRATTVPVMPVNTVAAPAPVPAEVGLGALNLSGEFMAVMHEMIRKEVRSYMEQQKNGMMCFQGMEMMEGFRNVSVKRIGISRVDS

SEQ ID NO: 96 - DNA, 861 bp, Oryza sativa
atggcggttccggtggtggagcagccggtgcaggcggcggcgacagactggatggggaggctgcaggtgacggcggaagggcttcgggatatcggcgctctagttgcagcggctgcgacgcgcatccaggccgcgcgcgccgcgctcggcgaggccgccgggctgatcggcgaggacgccagcgccgccgagaccctcgacgccgacgtgtggtccgccctcgcgcacgcaggccaggccccgatcccggacgccaccgtcgacgcggccgcgaagctcctcgccaccgtgtcctccggggcgccgctgctcccgggagccatccgcgccgccggggacctcatctccaccgtgttcgagatcgagatcgatatcgacgaccaggcggcggcggcggcgcccaccgggctactcagtgaggccatccgtgatctctcggtcgccttcggcttggggagcgtccacaacaacgtcgagttccacttcctcacgtgcgccccgtacctccacgttcgagccggcgacctcaccgacctgacgtggtttgcgtggagcaagcagacggagcgggccaagaagttggcgaccgaggcggagctgtggatcaacgccgcggcctgggaggccaaggatgcggcggagcgcgcccgctcccactccctggttcagtcgccggagcgcaacgagcacatgggggagctccaggtgagcctgctcatggccaccaggtacgccgacaaggcgctcgcggcggtggacatggtgcgcgacgcggtggagtcgatggaccagacgctccatcaagccatcggcaacgcccacatccctgatccctatcaccctatgccaatatggctatga

SEQ ID NO: 97 - protein, 286 aa, Oryza sativa
MAVPVVEQPVQAAATDWMGRLQVTAEGLRDIGALVAAAATRIQAARAALGEAAGLIGEDASAAETLDADVWSALAHAGQAPIPDATVDAAAKLLATVSSGAPLLPGAIRAAGDLISTVFEIEIDIDDQAAAAAPTGLLSEAIRDLSVAFGLGSVHNNVEFHFLTCAPYLHVRAGDLTDLTWFAWSKQTERAKKLATEAELWINAAAWEAKDAAERARSHSLVQSPERNEHMGELQVSLLMATRYADKALAAVDMVRDAVESMDQTLHQAIGNAHIPDPYHPMPIWL

SEQ ID NO: 98 - DNA, 948 bp, Oryza sativa
atggcggccgctgctcttctctcccctcctccctctccctctccctcccccaccccgtcctcgctccaccccaggcaagccctccgcttcgcggttggcacaggaggaggagggcgcgcgcgcgccacgtcgacggggacgagacgccgcgcggcgctcgtcccgtgctcctccagcgtgagcgcgcgcggccccgcttcgggaggcgacgggttggccctggagaggaggcgcctgctgctgtccggcctcgtgtcttcgttcgtgctcgtcctcccggtttcagattcgcatgctgttgccgagatggatgaagatgtgaaaatggctacgctagttgacccgatcaatgcatattcttttctttacccagttgaattgccaggaaagaaattcacattcaaatgggtagaatccagaaaaccagaacgatattcctctgctgcaccactatctcctgatgcacggcaacgtattgtatcggagcgagttgacatgatacataacgttgtcatctcagtctcgattgggccgccaaattcacgttttccgccttccaaggacaagagcaagtgggatccaaaagatgttgctgattggattttggctgaaaaatcttcactgaaggtgacgacaggccaacgcatgacagagagttctgtccttgatgcacactcttcagatgtcgatggagaaccatactggtactatgagtatctagttcggaaatctcctacacaatctgcaccagaaccaaatctgtttcgccataacgtagcctgcactgctgaacgagatggttacttgtactccttgaatgcttcaacactcagcaagcagtgggaatctatggggccttctttacagaaaacagtggcatcctttcacctcctaccccctacagaaaattatgttcctccataccaggatccatggagattttggtga

SEQ ID NO: 99 - protein, 315 aa, Oryza sativa
MAAAALLSPPPSPSPSPTPSSLHPRQALRFAVGTGGGGRARATSTGTRRRAALVPCSSSVSARGPASGGDGLALERRRLLLSGLVSSFVLVLPVSDSHAVAEMDEDVKMATLVDPINAYSFLYPVELPGKKFTFKWVESRKPERYSSAAPLSPDARQRIVSERVDMIHNVVISVSIGPPNSRFPPSKDKSKWDPKDVADWILAEKSSLKVTTGQRMTESSVLDAHSSDVDGEPYWYYEYLVRKSPTQSAPEPNLFRHNVACTAERDGYLYSLNASTLSKQWESMGPSLQKTVASFHLLPPTENYVPPYQDPWRFW

SEQ ID NO: 100 - DNA, 1311 bp, Zea mays
atgattgatcgcgaacgagcagaggaaatgcaagttaataatgaggcaccactagggtgcctcaagccaaatatttctcagtataactctccagagcagataggtggtgttgaagggtttcctgagaataatgaaaaaaggaatgatattgttgctgcggaaaaaatctgggaggcgactccaaaccaaggccttagcaggcccatctaccgacaagaattttatgcctggccatatatttattcagattatcaaatcgtgcgtcagccactaccttatggttttgacaaccaattttatcagataaatagggaccacggtttccctattgagaacagggttcaatatcttccattcaagatgctccctcaaggtcacccccatgatgcacagcttcaggaatttcagtattttgtggttattgactttgaagcaacctgtgacaaggtgaacaatccatttccacaagaaatcatcgagtttccatctgtcttggttaacagtgcaactggaaaactcgaagaatgcttccaaacatatgttcggccgacgtatcatcaatttttgactgatttttgcaaggagcttactggcatacaacagattcaggtggacagaggtgtgcctctaggtgaagccttactcatgcacgataaatggctagaggacaagggcatcaagaacacaaactttgctattgtgacctggtctaactgggactgccgtataatgctggagtcggaatgcagatttaagagaatcaggaagcccccttattttaacaggtggatcaacttgagggtaccgttccaggaagtgtacggggacgtccgctgcaacctgaaggaggcagtccagctggccgggctcacatgggaggggcgcgctcactgcgggctcgacgacgcccgcaacaccgctcgcctcctggcacttctgatgcaccggggcttcaagttctccatcaccaactcactggtgtggcaacctgccgccgctccgcagtcaaccgccgccacctgccacttctccccggaccgctctccggacccagtcgtcgtccagctccagcaccagcagcagcagcacaagccgaaggaggcgctggggtctcccgcgtcgctggtgaacccatcgtacgccacccccgcgggagggaaggacagagccatgtactgctactgcggggtgctgagccggtggagcgtcgtgcgcaagccgggacccatgcaggggcgctacttcttcgggtgcgggaactggaccgccacccggcgcgccatctgcccgtacttcgcatgggcctcgtga

SEQ ID NO: 101 - protein, 436 aa, Zea mays
MIDRERAEEMQVNNEAPLGCLKPNISQYNSPEQIGGVEGFPENNEKRNDIVAAEKIWEATPNQGLSRPIYRQEFYAWPYIYSDYQIVRQPLPYGFDNQFYQINRDHGFPIENRVQYLPFKMLPQGHPHDAQLQEFQYFVVIDFEATCDKVNNPFPQEIIEFPSVLVNSATGKLEECFQTYVRPTYHQFLTDFCKELTGIQQIQVDRGVPLGEALLMHDKWLEDKGIKNTNFAIVTWSNWDCRIMLESECRFKRIRKPPYFNRWINLRVPFQEVYGDVRCNLKEAVQLAGLTWEGRAHCGLDDARNTARLLALLMHRGFKFSITNSLVWQPAAAPQSTAATCHFSPDRSPDPVVVQLQHQQQQHKPKEALGSPASLVNPSYATPAGGKDRAMYCYCGVLSRWSVVRKPGPMQGRYFFGCGNWTATRRAICPYFAWAS

SEQ ID NO: 102 - DNA, 864 bp, Sorghum bicolor
atgtttgatttctttgtggtgatcgattttgaggcgacctgccaggaaggctcggtgatctacccacaggagatcatcgagttcccgtccgtcctcgtcgacggcgccaccggccggacgctgtccaccttccgcacctatgtccgccctcggcaccatccccgtctcaccgatttctgccgcgatctcaccggaatcacccagggagacgttgacgctggagtcagcctcgctgaggcactagagatgcacgaccactggctcgaagcacatggtgctaagctgggcaagctcgccgttgtaacctggggagattgggactgccgcacaatgctggagggggaatgccgcttcaaaggtatagaaaagcctcactatttcgatcactggattaatctaaggctgcccttctcggcagcgtttggtgtcggcaatgttcgtttcactctacaggatgcgattaggaaggcggggctgcagtgggagggccgcctccactgtggtctggatgatgctctaaacactgctcacctcctcgtcgagctcatgcggcggggaacccttctcaagatcacagcctctttggcaccaacgcagtcacctcctcgccctcaacctaaggcggcgttgccatgtgttggcccgcgctcggtggtgtcgacgctacctactcagcctcagcctcagcctcagctgccgctgccatgtgccgcagccactgggatggacacagtgccgtgctgcttctgtggtgtggccagcaagctaggcgtggtggccacgccgggccagatgcaggggcattacttctacggatgcggctggtggactccgatatgctctttcttcatgtgggcaacataa

SEQ ID NO: 103 - protein, 287 aa, Sorghum bicolor
MFDFFVVIDFEATCQEGSVIYPQEIIEFPSVLVDGATGRTLSTFRTYVRPRHHPRLTDFCRDLTGITQGDVDAGVSLAEALEMHDHWLEAHGAKLGKLAVVTWGDWDCRTMLEGECRFKGIEKPHYFDHWINLRLPFSAAFGVGNVRFTLQDAIRKAGLQWEGRLHCGLDDALNTAHLLVELMRRGTLLKITASLAPTQSPPRPQPKAALPCVGPRSVVSTLPTQPQPQPQLPLPCAAATGMDTVPCCFCGVASKLGVVATPGQMQGHYFYGCGWWTPICSFFMWAT

SEQ ID NO: 104 - DNA, 1245 bp, Glycine max
atgatggcccttgaaaattctgaaaatatgcaaataaactgtgaggcatctttaaaatgcctccagagcaagggatttccttgcaattttcaaagtaatgggaattctatggaaggctatacagagcttaaaaatgagcctggtactcaccctgctggggatgttgctgagcctaactgtcacttaggaagtgagtttcttgagccttccaatgaattccacacaaaacctacttatcaccaaaattacagcacctggacgccctgccattttaactctcacaaggtgcagcaatgccaaatgaatgcttttgagagccattattatccttatcctgttgagaacccacttcagtatgttcctattaatatggttgcacaaggttacccgcgtgagcaatatcaggaatttcagtattttgtggtgatagactttgaggctacttgtgacaaagataaaaatccccaccctcaagaaataattgagtttccatctgttatagtgagtagcatcactggccagctggaagcgtgttttcaaacatatgtgaggcccacctgcaatcagcttctgactgatttctgcaaggatctgactggtatccagcaaattcaggtggacagaggtgttacattgagtgaggctctacttaggcatgacaaatggcttgagaagaagggaataaagaattccaactttgctgtggttacatggtccaactgggattgtcgggtgatgcttgaatctgagtgccgattcaagaagatacggaagcctccttacttcaaccgctggatcaacttgaggattcctttccgtgaggtatttggtgctgtgaggtgcaatctaaaggaagctgttgagatagccggcttggcctggcagggacgtgcacattgcggccttgatgatgccaaaaatactgctcacctattggcacttctcatgcaccggggttttaaattttccattaccaattccataatgtggcagacagctgatcgaccactgatgtggaaacagtcaccagaacaaccaattgttttcccgcattccccctataaagcaaaggatatcactatccccgtggttcagtatcaccctttctgcttttgtggggtgaaaagcagcaggggcatggtgaggaagccgtgtcccaagcaagggagtctcttctttggatgtggaaattggactgcgactagaggtgcttgctgccgttactttgaatgggcttctaactga

SEQ ID NO: 105 - protein, 414 aa, Glycine max
MMALENSENMQINCEASLKCLQSKGFPCNFQSNGNSMEGYTELKNEPGTHPAGDVAEPNCHLGSEFLEPSNEFHTKPTYHQNYSTWTPCHFNSHKVQQCQMNAFESHYYPYPVENPLQYVPINMVAQGYPREQYQEFQYFVVIDFEATCDKDKNPHPQEIIEFPSVIVSSITGQLEACFQTYVRPTCNQLLTDFCKDLTGIQQIQVDRGVTLSEALLRHDKWLEKKGIKNSNFAVVTWSNWDCRVMLESECRFKKIRKPPYFNRWINLRIPFREVFGAVRCNLKEAVEIAGLAWQGRAHCGLDDAKNTAHLLALLMHRGFKFSITNSIMWQTADRPLMWKQSPEQPIVFPHSPYKAKDITIPVVQYHPFCFCGVKSSRGMVRKPCPKQGSLFFGCGNWTATRGACCRYFEWASN

SEQ ID NO: 106 - DNA, 1269 bp, Oryza sativa
atgcctccctgtctccggcggtggccgaccaccgctcgtccccggcagccgcgaccgcctccctcctccccttctgccgctccaccccgctctccgcgtaagcaacgcgaacccgcggctacaacccattttcttggctccagtggtgcatgtgacaacacggtgagacgttgtgtgtgggtgggtgggtgcaggggcggtggtggcgtcgcgatgggggaggacgcgccgatgaccgcgaggtggccgccggcggcggcggcgaggctgccgccgttcaccgcggcgcagtacgaggagctggagcagcaggcgctcatatacaagtacctggtggcaggcgtgcccgtcccgccggatctcgtgctccccatccgccgcggactcgactccctcgccgcccgcttctacaaccatcccgcccttggatatggtccgtacttcggcaagaagctggacccagagccagggcggtgccggcgtacggacggcaagaaatggcggtgctcgaaggaggccgcgccggattccaagtactgcgagcgccacatgcaccgcggccgcaaccgttcaagaaagcctgtggaaacgcagctggtcgcccagtcccaaccgccctcatctgttgtcggttctgcggcggcgccccttgctgctgcctccaatggcagcagcttccaaaaccactctctttaccctgctattgccggcagcaatggcgggggcggggggaggaacatgcccagctcatttggctcggcgttgggttctcagctgcacatggataatgctgccccttatgcagctgttggtggtggaacaggcaaagatctcaggtatactgcttatggcacaagatctttggcggatgagcagagtcaactcattactgaagctatcaacacatctattgaaaatccatggcggctgctgccatctcagaactcgccatttcccctttcaagctattctcagctgggggcactaagtgaccttggtcagaacacccccagctcactttcaaaggttcagaggcagccactttcgttctttgggaacgactatgcggctgtcgattctgtgaagcaagagaaccagacgctgcgtcccttctttgatgagtggccaaagggaagggattcatggtcagacctcgctgatgagaatgctaatctttcgtcattctcaggcacccaactgtcgatctccataccaatggcatcctctgacttctcggcggccagttctcgatcaactaatggtgactga

SEQ ID NO: 107 - protein, 422 aa, Oryza sativa
MPPCLRRWPTTARPRQPRPPPSSPSAAPPRSPRKQREPAATTHFLGSSGACDNTVRRCVWVGGCRGGGGVAMGEDAPMTARWPPAAAARLPPFTAAQYEELEQQALIYKYLVAGVPVPPDLVLPIRRGLDSLAARFYNHPALGYGPYFGKKLDPEPGRCRRTDGKKWRCSKEAAPDSKYCERHMHRGRNRSRKPVETQLVAQSQPPSSVVGSAAAPLAAASNGSSFQNHSLYPAIAGSNGGGGGRNMPSSFGSALGSQLHMDNAAPYAAVGGGTGKDLRYTAYGTRSLADEQSQLITEAINTSIENPWRLLPSQNSPFPLSSYSQLGALSDLGQNTPSSLSKVQRQPLSFFGNDYAAVDSVKQENQTLRPFFDEWPKGRDSWSDLADENANLSSFSGTQLSISIPMASSDFSAASSRSTNGD

SEQ ID NO: 108 - DNA, 1260 bp, Zea mays
atggcgatgccgtatgcctctctttccccggcaggcgccgccgaccaccgctcctccacagccacggcgtccctcgtccccttctgccgctccactccgctctccgcgggcggcgggctgggcgaggaggacgcccaggcgagcgcgaggtggccggccgcgaggccggtggtgccgttcacgccggcgcagtaccaggagctggagcagcaggcgctcatatacaagtacctggtggcgggcgtgcccgttccgccggatctcgtggttccaatccgccgcggcctcgactccctcgcaacccgcttctacggccaacccacactcgggtacggaccgtacctggggaggaaactggatccggagcccggccggtgccggcgaacggacggcaagaagtggcggtgctccaaggaggccgccccggactccaagtactgcgagcgccacatgcaccgcggccgcaaccgttcaagaaagcctgtggaaacgcagctcgcgccccagtcccaaccgcccgccgccgcagccgtctccgccgctccgcccctggcagccgccgccgccgccaccaccaacggcagcggcttccagaaccactctctctacccggccatcgccggcagcactggtggtggaggaggagttggcgggtccggcaatatctcctccccgttctcctcgtcgatggggggatcgtctcagctgcacatggacagtgctgccagctactcctacgcagctcttggtggtggaactgcaaaggatctcaggtacaacgcttacggaataagatctctggcggacgagcacaaccagctgatcgcagaagccatcgactcgtcgatagagagccagtggcgcctccccagctcgtcgttcccgctctcgagctacccacatctcggggcgctgggcgacctgggcggccagaacagcacggtgagctcgctgccgaagatggagaagcagcagccgccctcgtccttcctagggaacgacaccggggccggcatggccatgggctccgcctccgcgaagcaggagggccagacgctgcggcacttcttcgacgagtggcccaaggcgcgggactcctggccgggcctctccgacgagaccgccagcctcgcctcgttccccccggcgacccagctgtcgatgtccatacccatggcgtcctccgacttctccgtggccagctcccagtcgcccaacggtgagtcgcgtacgttcctgctggccacggaccgaaggtga

SEQ ID NO: 109 - protein, 419 aa, Zea mays
MAMPYASLSPAGAADHRSSTATASLVPFCRSTPLSAGGGLGEEDAQASARWPAARPVVPFTPAQYQELEQQALIYKYLVAGVPVPPDLVVPIRRGLDSLATRFYGQPTLGYGPYLGRKLDPEPGRCRRTDGKKWRCSKEAAPDSKYCERHMHRGRNRSRKPVETQLAPQSQPPAAAAVSAAPPLAAAAAATTNGSGFQNHSLYPAIAGSTGGGGGVGGSGNISSPFSSSMGGSSQLHMDSAASYSYAALGGGTAKDLRYNAYGIRSLADEHNQLIAEAIDSSIESQWRLPSSSFPLSSYPHLGALGDLGGQNSTVSSLPKMEKQQPPSSFLGNDTGAGMAMGSASAKQEGQTLRHFFDEWPKARDSWPGLSDETASLASFPPATQLSMSIPMASSDFSVASSQSPNGESRTFLLATDRR

SEQ ID NO: 110 - DNA, 1137 bp, Sorghum bicolor
atggcgatgccctttgcctccctgtctccggcagccgaccaccgcccctcctccctcctccccttctgccgcgccgcccctctctccgcggtgggagaggacgccgcgcagcaccaccagcagcagcagcagcagcacacgatgagcggcaggtgggcggcgaggccggcgctcttcacggcggcgcagtacgaggagctggagcaccaggcgctcatatacaagtacctcgtcgccggcgtgcccgtcccgccggacctcctcgtccccttacgccgaggcttcgtctaccaccagcccgcccttggctatgggacctacttcggcaagaaggtggacccggagcccgggcggtgccggcgtacggacggcaagaagtggcggtgctccaaggaggctgccccggactccaagtactgcgagcgccacatgcaccgcggccgcaaccgttcaagaaagcctgtggaagcgccgctcgtgcccccgccgcacgccccccagcagcagcagcagcagcagcagccgccgcccgcccccgttgctggcttccagaaccactcgctgtacccgtcggtcctcgccggcaacggcggcgtcggggtaggaggtggtggcggtggcacgttcggcatggggcccacctctcagctgcacatggacagtgccgctgcttacgcgactgctgctggtggagggagcaaagatctcaggtactctgcatacggggcgaaatctctgtctgatgatcacagccagctgttgcccggcggcatggatccgtccatggacaactcatggcgcctgttgccatcccaaaccaccacatttcaagccacaagctaccctgtgtttggcacgctgagcggtctggatgagagcaccatcgcctcactgccgaagacgcagagggagcctctctctttcttcgggagcgactatgtgaccgccaagcaggagaaccagacgctgcgccctttcttcgacgaatggcccaagtcaagggagtcgtggccagagctggctgaggacaaccaccttggcttctcagccacccagctctccatctccattcccatggcgacctcagacttctccaacaccagctccagatcgccgaacggaataccgtcaagatga

SEQ ID NO: 111 - protein, 378 aa, Sorghum bicolor
MAMPFASLSPAADHRPSSLLPFCRAAPLSAVGEDAAQHHQQQQQQHTMSGRWAARPALFTAAQYEELEHQALIYKYLVAGVPVPPDLLVPLRRGFVYHQPALGYGTYFGKKVDPEPGRCRRTDGKKWRCSKEAAPDSKYCERHMHRGRNRSRKPVEAPLVPPPHAPQQQQQQQQPPPAPVAGFQNHSLYPSVLAGNGGVGVGGGGGGTFGMGPTSQLHMDSAAAYATAAGGGSKDLRYSAYGAKSLSDDHSQLLPGGMDPSMDNSWRLLPSQTTTFQATSYPVFGTLSGLDESTIASLPKTQREPLSFFGSDYVTAKQENQTLRPFFDEWPKSRESWPELAEDNHLGFSATQLSISIPMATSDFSNTSSRSPNGIPSR

SEQ ID NO: 112 - DNA, 1593 bp, Arabidopsis thaliana
atggatcttggagttcgtgtttctggtcatgaaaccgtttcttctccgggtcaaactgaactcggatctggtttcagtaacaagcaagaaagatccggtttcgatggtgaagattgctggagaagttcaaagctctcacgaacatcaactgatggattctcttcttcccctgcctctgctaaaacgctgtcgtttcatcaaggcatccctttactgagatctaccactattaatgatcctcgtaaaggacaagaacacatgcttagcttctcttctgcttcaggcaaatcagatgtctcaccttatcttcagtactgtagaaactcaggatatggtttaggaggaatgatgaacacaagcaacatgcatggaaacttgttgacaggagtaaaaggacctttttcattgactcagtgggcagagctagagcaacaggcgttgatctataagtatatcacagccaatgtccctgttccatctagtttacttctctctctcaagaaatcttttttcccttatggttccttgcctcctaattcttttggatggggctcttttcatctgggcttttccggtggtaacatggatcccgagccagggagatgtcgccggacagatggaaagaaatggcggtgctcgagggacgctgttcccgatcaaaagtactgtgaacgacatattaacagaggccgccatcgttcaagaaagcctgtggaaggccaaaatggccacaatactaatgctgccgccgctgcttctgctgctgccgcttctaccgctgctgctgtgtccaaagcggcagcggggacttcagctgttgcgatgcgtggatcagataataacaatagccttgccgctgctgttggaacacaacatcataccaataatcaatctacagattctttggctaacagagttcaaaattctcgaggggcttcggtttttcctgccacgatgaacttacagtcgaaggaaactcatccgaaacaaagcaataatccctttgaattcggactcatctcttctgattcgttacttaatccgtcgcataaacaagcctcgtatgcaacctcttccaaaggctttggatcgtatcttgacttcggcaaccaagccaagcacgcggggaatcacaacaatgtcgattcttggcccgaagagctgaaatcggattggactcagctctcaatgtcaatccctatggctccatcttcccctgttcaagataaacttgcactctcacctttaaggttatcgcgtgagtttgaccccgcgatccacatgggattaggcgtcaacaccgagtttcttgaccccgggaaaaagacgaataactggataccaatctcctggggtaataacaactccatgggaggtccactcggcgaggtactaaacagcacgaccaatagtcccaagtttggttcctctccaacaggcgtcttgcaaaagtcgacatttggttctctttctaacagcagctcggcaagcagcaccatcattggcgataacaacaataagaacggtgatggaaaagatccgcttggcccgaccacgctgatgaatacttctgctactgctccttctctgtga

SEQ ID NO: 113 - protein, 530 aa, Arabidopsis thaliana
MDLGVRVSGHETVSSPGQTELGSGFSNKQERSGFDGEDCWRSSKLSRTSTDGFSSSPASAKTLSFHQGIPLLRSTTINDPRKGQEHMLSFSSASGKSDVSPYLQYCRNSGYGLGGMMNTSNMHGNLLTGVKGPFSLTQWAELEQQALIYKYITANVPVPSSLLLSLKKSFFPYGSLPPNSFGWGSFHLGFSGGNMDPEPGRCRRTDGKKWRCSRDAVPDQKYCERHINRGRHRSRKPVEGQNGHNTNAAAAASAAAASTAAAVSKAAAGTSAVAMRGSDNNNSLAAAVGTQHHTNNQSTDSLANRVQNSRGASVFPATMNLQSKETHPKQSNNPFEFGLISSDSLLNPSHKQASYATSSKGFGSYLDFGNQAKHAGNHNNVDSWPEELKSDWTQLSMSIPMAPSSPVQDKLALSPLRLSREFDPAIHMGLGVNTEFLDPGKKTNNWIPISWGNNNSMGGPLGEVLNSTTNSPKFGSSPTGVLQKSTFGSLSNSSSASSTIIGDNNNKNGDGKDPLGPTTLMNTSATAPSL

SEQ ID NO: 114 - DNA, 858 bp, Glycine max
atgaacaacagcagtggcggaggaggacgaggaactttgatgggtttgagtaatgggtattgtgggaggtcgccattcacagtgtctcagtggcaggaactggagcaccaagctttgatcttcaagtacatgcttgcgggtcttcctgttcctctcgatctcgtgttccccattcagaacagcttccactctactatctcgctctcgcacgctttctttcaccatcccacgttgagttactgttccttctatgggaagaaggtggaccctgagccaggacgatgcaggaggactgatggaaaaaagtggaggtgctccaaggaagcatacccagactccaagtactgcgagcgccacatgcaccgtggccgcaaccgttcaagaaagcctgtggaatcacaaactatgactcactcatcttcaactgtcacatcactcactgtcactgggggtagtggtgccagcaaaggaactgtaaatttccaaaacctttctacaaatacctttggtaatctccagggtaccgattctggaactgaccacaccaattatcatctagattccattccctatgcgattccaagtaaagaatacaggtacagacatctttatcatattttgcacccattgtttgcttggaatttgaaaaatacttccactgtaaagaatagaatagaaaatatatctttaattaaagttgttttgcttggcatattcaatggaactgcctttttttataactaccgtcagccttttttcattaaaaatcctgaactcttattttgtttatttccggtttctgttttatattcttttctagtgttgggtgatggtctgacatgtggaatagccgaatag

SEQ ID NO: 115 - protein, 285 aa, Glycine max
MNNSSGGGGRGTLMGLSNGYCGRSPFTVSQWQELEHQALIFKYMLAGLPVPLDLVFPIQNSFHSTISLSHAFFHHPTLSYCSFYGKKVDPEPGRCRRTDGKKWRCSKEAYPDSKYCERHMHRGRNRSRKPVESQTMTHSSSTVTSLTVTGGSGASKGTVNFQNLSTNTFGNLQGTDSGTDHTNYHLDSIPYAIPSKEYRYRHLYHILHPLFAWNLKNTSTVKNRIENISLIKVVLLGIFNGTAFFYNYRQPFFIKNPELLFCLFPVSVLYSFLVLGDGLTCGIAE

SEQ ID NO: 116 - DNA, 2130 bp, Oryza sativa
atgcatcaacataggatcactatgctatcttcctcagaaacatgtcaccttggttctagttcgaacaaccaagccatggatcagcagaatttactgcccagcaaccccaccgcagatgaacagaatttacttccaaatactctagaggatgatgattacccacattatttacttggtagtcatgaggtggaaatgccaaatggaagcgtgattggtcagcaaaatacaagcttgaacttatgggattcagctggatctagctcgatgggctgtgtagctgatcatgatagtctttttgaggccaaaagggaacattttgctcctgctttgtctatccgagctcccttaattattggagggagaagacatgaaggcagtagttcattgccttcacagagcttaaacttagaccttaatcttaatcaggctgatcagtttgattctgaggatgttgatatgattcagagtaatggacaaccagggataaacgcttttcctctcaacaggggcctttccattcctgagcatgttctgcgccatacaaattcttccagtgctacaggaaatccttcacaggttgcaagcttttctgatggaatgacaggccaagaagttaacctgtttggtgggcatcgttcatcttgcaagagaaagaatattgatgggagtcttgcagagtcttctgccaatggtagttcacgtaataatcagcgaaataatattatactggaaccttctccatccagtcatgaaagcacttctggtttaactgcacctgcccctacaaaccatgttttttcatactctcctgtggaacagctaaaccagaataccaatatgtctgcaaatgctatgttgtctgatcattattcactatatggtgatcatgagcgtgagagatttctgaggaatacccggatgagaacaagccctaatgagtatgatcaatcatcgtccaatctcttgcctgaaggaagtctcaggtgttctgtttatcagcctactcagcaacagtctttgtttattccagtacaacctagagcatcgagctcttcaacaagttctcttagtcggccttatgtgcctgctgtcactcaattctcacaaaatttgcaccgtgctccatcaagtggcaattttggttcgagaatagggatttttcctagttctgctgatacaacaaaccagttatcttcacaagatcccaacaggagctcggtgagaggcaattttcctgagccccttctgttaggttcttctctgtttccttctgactcggcagaattgctatctatgccgggaggcagaagcaaccaacaaaattccagctccacaattcgaactgctgtaaatataggagctcaacagattgctgggttaaatgcatcccagcctacttcaagctcaaggggttcggttgatattgttaggagatccttgcaggctgctagcgttcctcagtccagaggttcaagcattacatcacagcagcaacgtgggcattcatccacatcacatgagattcgaagccatcaacctggatccagctctcgtgccaatcagcagcattatgtcagggctgttcctcactctgtagataggcaaaactcgaattacttggacctgcagtcttttatgcaaagcattgctgcttcaagagacggaattaggacagtctcagagtctgccaatcaacttgtgcatcttcgcaatgttgttgaacaaattcgtcagggaagaggtggaaggtttgaggatcctaattttgaacgtgcactttttgcaaggcgtgccagtttaattgacagacatcgtgacatgcggcttgatgtggataatatgtcatatgaggaattgttggcacttggtgaacgcattgggtatgtaaacactggacttagtgaggataaaattaggactggtttgaagcaatggaaatatgtgagcataccgattgaagaacctctaactggtgttgaaccatgctgtatttgccaggaagaatatgccgaaggtgaggacatgggcagactagactgtgggcatgacttccacaccgcatgcatcaaacaatggctggttataaaaaatctgtgccctatctgtaaaaagacaggactgggcacttaa

SEQ ID NO: 117 - protein, 709 aa, Oryza sativa
MHQHRITMLSSSETCHLGSSSNNQAMDQQNLLPSNPTADEQNLLPNTLEDDDYPHYLLGSHEVEMPNGSVIGQQNTSLNLWDSAGSSSMGCVADHDSLFEAKREHFAPALSIRAPLIIGGRRHEGSSSLPSQSLNLDLNLNQADQFDSEDVDMIQSNGQPGINAFPLNRGLSIPEHVLRHTNSSSATGNPSQVASFSDGMTGQEVNLFGGHRSSCKRKNIDGSLAESSANGSSRNNQRNNIILEPSPSSHESTSGLTAPAPTNHVFSYSPVEQLNQNTNMSANAMLSDHYSLYGDHERERFLRNTRMRTSPNEYDQSSSNLLPEGSLRCSVYQPTQQQSLFIPVQPRASSSSTSSLSRPYVPAVTQFSQNLHRAPSSGNFGSRIGIFPSSADTTNQLSSQDPNRSSVRGNFPEPLLLGSSLFPSDSAELLSMPGGRSNQQNSSSTIRTAVNIGAQQIAGLNASQPTSSSRGSVDIVRRSLQAASVPQSRGSSITSQQQRGHSSTSHEIRSHQPGSSSRANQQHYVRAVPHSVDRQNSNYLDLQSFMQSIAASRDGIRTVSESANQLVHLRNVVEQIRQGRGGRFEDPNFERALFARRASLIDRHRDMRLDVDNMSYEELLALGERIGYVNTGLSEDKIRTGLKQWKYVSIPIEEPLTGVEPCCICQEEYAEGEDMGRLDCGHDFHTACIKQWLVIKNLCPICKKTGLGT

SEQ ID NO: 118 - DNA, 1968 bp, Zea mays
atgcaaggacagaggaattctgtggagcattttgctgatgtttttggattcgacattgcatctagttcaggcaaccctgtgatggatcagcagtcatattggaacaatgttcttggatcagtagaatcgcagaatcttcaaggttatcagatgaatcacagtgatgctgccatgccatatgggaatgagacacagcaagatggtacatttcttggtttctgggaatcaggcgaagcaagtgcaagtggcagctctaacaatgccaaaacagagcatcttagtattggcggtggtctgaggattggtgaaaggcgactggtagctgacaatggcatttctctggatgtggatatcaaccttaacgccaacgttaacgatctatgtggtcaaagttcaaatgttaactgtacatctcagggccctgagcactatggtggcggtgatcgtagtgttgtaaattctcagccaactgacctgagattacacccatacaggactttcctactagatgcagagcaagcagattcttttactttgaaccctagtgaaaaccctttgtgtgatttttcactgatgcaagaaagcattgaccaaagacccggtagttccctggatggacgccggctagcctgcaagagaaaaaatgttgaaggacccaatggccagagttcagcaggtgctagcactagtttttcccacaggaacgacaatgctttccataacattgcttcttcaagttacaatcctgcacctatcagaaatccgtcctcacccaattgcttgccggttccaagttctatcgatgatcaactcccacgatatggaactaatgcagggctctcagccggtacctatgaccttaatggaggggtcaacaatgctgggaattcgcagagaagtttccgggcaagaattactacgtctcaacagattgctccctgtagtgtatggccctcttcaaatgctatcagacttcctaattcatggaatcatcagccacctcattttcaaagtgcatttgatgatacccaggaggttattcctgtggtcagcagcctcaacttgcaataccagcatccagtgaatgtttctagtgtgccaccggctgcaaaccgtttcactggccatggagcttcatcatcgagagctggcagtttggagaacagaattcttggtagtcaagaggctcctacaaggaatgtggtgcctgccaactactctgatttagttcccccgtctgtagtagacccgagacgtttgctgccagaaccatctaattggagttctgatgtccgaggcactgcaatatcaggaagtattcctcctgtatcaagagctaataacagtgcaactgttaatccgccagcaggattcagtcaccaaaacctcacccggcgccatcctcgaaatttatcagaggagattggtcggctatctggagcacttcgcggccatcaacccccacgcttaaggccggggtttctgttggagcgccagggcgatggtgtttggggtgttcctttatcaacaaggggtagagaaggaagaaggttaatggagattcggaatgcacttgaaatgatccatagaggggagaatgtaaggcttgagtctatcttctatggcggtgttgacattcacgatagacacagggacatgcgccttgacattgacaatatgtcctatgaggagctattagcactcgaggaaagaataggaaatgtcggcactggcctcagcgaggaagctgtgataaggttgctcaaacaaaggaaattttcatcttggacactaaaagcatctttggaccctgaaccatgttgtatctgccaggaggagtacgctgatggagacgacctcgggaagctggactgcgggcacgacttccacgctggctgcatcaagcaatggctggtggtgaagaacgtgtgccccatctgcaagagcaccgcattgaagaagacctga

SEQ ID NO: 119 - protein, 655 aa, Zea mays
MQGQRNSVEHFADVFGFDIASSSGNPVMDQQSYWNNVLGSVESQNLQGYQMNHSDAAMPYGNETQQDGTFLGFWESGEASASGSSNNAKTEHLSIGGGLRIGERRLVADNGISLDVDINLNANVNDLCGQSSNVNCTSQGPEHYGGGDRSVVNSQPTDLRLHPYRTFLLDAEQADSFTLNPSENPLCDFSLMQESIDQRPGSSLDGRRLACKRKNVEGPNGQSSAGASTSFSHRNDNAFHNIASSSYNPAPIRNPSSPNCLPVPSSIDDQLPRYGTNAGLSAGTYDLNGGVNNAGNSQRSFRARITTSQQIAPCSVWPSSNAIRLPNSWNHQPPHFQSAFDDTQEVIPVVSSLNLQYQHPVNVSSVPPAANRFTGHGASSSRAGSLENRILGSQEAPTRNVVPANYSDLVPPSVVDPRRLLPEPSNWSSDVRGTAISGSIPPVSRANNSATVNPPAGFSHQNLTRRHPRNLSEEIGRLSGALRGHQPPRLRPGFLLERQGDGVWGVPLSTRGREGRRLMEIRNALEMIHRGENVRLESIFYGGVDIHDRHRDMRLDIDNMSYEELLALEERIGNVGTGLSEEAVIRLLKQRKFSSWTLKASLDPEPCCICQEEYADGDDLGKLDCGHDFHAGCIKQWLVVKNVCPICKSTALKKT

SEQ ID NO: 120 - DNA, 1983 bp, Sorghum bicolor
atgcaagggcagaggaattctatggagcattatgctgatgtttttggattcgacattgcatctagttcaggcaaccctgtgatggaccagcagtcatattggaataatgttcttggatcagtagaatcacagaatcctcaaggttatcagatgaatcacagtgatgccgccatgccatatggaaatgaggcacagcaagatggtacatttcttggtttctgggaatcaggcgaagcaagttcaagtggcagcgcactaaactatggcagctccaacaatgtcaaaacagagcatcttaacattggcggtggactgaggattggtgaaaggcaactggtagctgacaatggcatttctctggatgtggatatcaaccttaacgccaacgttaacgatctatgtggccaaagttcaaatgttaactgtacctctcaaggtcctgagcagtatggtggcagtgatcgtaatggtataaattctcagccaactgacctgagattacacccatataggacattcctgctaggtgcagagcaagcagactcttttactttgaatcctagtgaaaatcctttgagtgatttttcactaatgcaagaaagcattgatcaaagagcaggtagttccctggatggtcgccggctagcgtgcaagagaaaaaatattgaaggagccaatggccagagttcagcaggtgctagcacaagtttttccctcaggaacgataatgctttctataacattgcttcttcaagttacaatcctgcacctatcagaaattcatcctctcccaattgcttgccagttccaagttctattgaggatcaactcccacaatatggaactaatgcagggctctcagccggtacctatgaccttaatggaggggtccacaatgctgggaattcgcagagaagtttccgggcaagaactaccacgtctcaacagattgctccctgtagtgtatggccctcttcaaatgctatcaggctttctaattcatggaatcaccagccacctcattttcaaaatgcatttgatggtccccaggaagttattcctgtggtcagcagcctcaacttgcaataccagcatccagtgaatgtttctggtgtgccacaggctgcaaaccgtttcactggccatggggcttcatcatcgagagctacgagtttggagaacagaaatcttggtagtgaagaggttactaggaggaatgtggtgcctaccaactacactgatttagttcccccgtctgcagtagacctgagacgtttggtgccagaaccatctaactggagttctgatgtccgaggcactgcaatatcaggaagtattcctcctgtatcaagagctaataacagttcagcagctaatccaccagcaggattcaatcaccaaaacctcacccgtcgccatcctcgaaatttgtcagaggagattggtcgtctatccggagcacttcgcggccatcaacccccacgcttaaggtcagggtttctgttggagcgccagggcgatggtgtttggggtgttcctttatcaacaaggggtagggaaggaagaaggctaatggagattcggaatgcacttgaaatgattcatagaggggagaatgtaaggcttgagtctatcttctatggtggcgttgaaattcatgacagacacagggacatgcgccttgacattgacaatatgtcctatgaggaactattagcacttgaggaaagaataggagatgttagcactggcctcagcgaggaagctgtgataaagttgctcaaacaaaggaaattttcatcatggagactgaaagcatctttggaccctgaaccgtgttgtatctgccaggaggagtacgttgatggagacgatcttgggaggctggactgcgggcacgacttccacgcttgctgcatcaagcaatggctggtggtgaagaacgtgtgccccatctgcaaaaacaccgcattgaagacctga

SEQ ID NO: 121 - protein, 660 aa, Sorghum bicolor
MQGQRNSMEHYADVFGFDIASSSGNPVMDQQSYWNNVLGSVESQNPQGYQMNHSDAAMPYGNEAQQDGTFLGFWESGEASSSGSALNYGSSNNVKTEHLNIGGGLRIGERQLVADNGISLDVDINLNANVNDLCGQSSNVNCTSQGPEQYGGSDRNGINSQPTDLRLHPYRTFLLGAEQADSFTLNPSENPLSDFSLMQESIDQRAGSSLDGRRLACKRKNIEGANGQSSAGASTSFSLRNDNAFYNIASSSYNPAPIRNSSSPNCLPVPSSIEDQLPQYGTNAGLSAGTYDLNGGVHNAGNSQRSFRARTTTSQQIAPCSVWPSSNAIRLSNSWNHQPPHFQNAFDGPQEVIPVVSSLNLQYQHPVNVSGVPQAANRFTGHGASSSRATSLENRNLGSEEVTRRNVVPTNYTDLVPPSAVDLRRLVPEPSNWSSDVRGTAISGSIPPVSRANNSSAANPPAGFNHQNLTRRHPRNLSEEIGRLSGALRGHQPPRLRSGFLLERQGDGVWGVPLSTRGREGRRLMEIRNALEMIHRGENVRLESIFYGGVEIHDRHRDMRLDIDNMSYEELLALEERIGDVSTGLSEEAVIKLLKQRKFSSWRLKASLDPEPCCICQEEYVDGDDLGRLDCGHDFHACCIKQWLVVKNVCPICKNTALKT

SEQ ID NO: 122 - DNA, 2001 bp, Arabidopsis thaliana
atgcaaggtccacgaagcactggtgattcatcgactggaataaattatgcagatggagaacccatctgtagcaccaattcagagactacttccaataacatcctgaatccagtggatgttcagtttccaaacaatactacaggttcaggacgaccaacttacgcaagctctagctcgcatgttgttcaaaatcataactggtggagttttggtgaatccagctctagattggggccttctgatcatttaaattccaatggttcaaagactgatcgacagcttctctcagatggctatggatttgaggaagggcaatcaggtatgttgttacctggagagtcctttttgcgtgggtcaagctctagtcatatgttaagtcatgtaaatctgggcaaggacatggacattggtagtggtctgcagacttctggggtcgttatccgccataataactgcgagacttcattgggaagctcaagtcaaaccgcagaggagagaagcagtggtccaggttcttcgttgggtggtctaggttcatcctgcaaaagaaaggctcttgaaggagctcctagccattctttccctggtgaaagtcatggttgcttttttcaaactgagaatggtgcttggaatgagggtcttgctcaatatgatgcttcaagtagcttaagtttgtctatgccctcacaaaattctccaaatgttaataatcagtctggtctaccagaaccaagatttggattgggtggtgggagagcagttacagcaagtgcttttccttctacaagaagcacggaaaccatctctagacctggcaggcggttaaatcctgggcagccaccagagtcagttgcattcagcttcacacagtccggtagttctgtgcgtcagcaacagcagttaccagcaacttctccttttgttgaccctctggatgcaagagcaataccagttacaggtagctcaagcagtggtgatggtcagccaagtatgatccaccttcctgcattgacaagaaatatacaccaatttgcttggagtgcttcgtctagttcgagagcaaacagtatgcctgaagagggattgtcaccatgggacgcgccaagaataaactcagagcagccagtctttactacacctgcaaatgaaacgagaaatccagtgcaggatcagttttgttggagtttcactcgtggaaaccctagtacatctggagattctccctttgttcctcgagcaggatcgagttcagggatccatggtttgcagccgaatcccacttgggttactccccataatcaatcaagaatatcagaagttgctccgtggtctttatttcctagtatcgaatctgaatctgctactcatggtgcttctcttccattactacccacagggccttctgtttcctcgaatgaagctgcagcgccgtctggatctagtagtcgtagtcatcgctctcgacaaagaagatcgggattattactggaaaggcaaaatgatcatctccatttgcgtcacttaggaagaagcttagctgctgataatgatggaaggaaccggctgatttccgagatacggcaggtgttgagtgccatgcgaagaggggagaatttacggtttgaggattatatggtattcgatccactgatctaccaaggtatggccgagatgcatgataggcatcgggatatgcgtcttgatgttgataacatgtcatatgaggagctattggcacttggggaacgcataggagatgtgagcactggtctaagcgaagaggtgatcctgaaagtaatgaaacagcacaaacatacatcatccgctgctggctctcaccaggacatggagccttgctgtgtctgtcaggaagagtatgcagaaggagatgatcttggaacactgggatgtggtcatgaatttcacactgcctgcgtcaagcaatggctgatgctcaagaatctctgcccaatttgtaagactgtggctttatcgacataa

SEQ ID NO: 123 - protein, 666 aa, Arabidopsis thaliana
MQGPRSTGDSSTGINYADGEPICSTNSETTSNNILNPVDVQFPNNTTGSGRPTYASSSSHVVQNHNWWSFGESSSRLGPSDHLNSNGSKTDRQLLSDGYGFEEGQSGMLLPGESFLRGSSSSHMLSHVNLGKDMDIGSGLQTSGVVIRHNNCETSLGSSSQTAEERSSGPGSSLGGLGSSCKRKALEGAPSHSFPGESHGCFFQTENGAWNEGLAQYDASSSLSLSMPSQNSPNVNNQSGLPEPRFGLGGGRAVTASAFPSTRSTETISRPGRRLNPGQPPESVAFSFTQSGSSVRQQQQLPATSPFVDPLDARAIPVTGSSSSGDGQPSMIHLPALTRNIHQFAWSASSSSRANSMPEEGLSPWDAPRINSEQPVFTTPANETRNPVQDQFCWSFTRGNPSTSGDSPFVPRAGSSSGIHGLQPNPTWVTPHNQSRISEVAPWSLFPSIESESATHGASLPLLPTGPSVSSNEAAAPSGSSSRSHRSRQRRSGLLLERQNDHLHLRHLGRSLAADNDGRNRLISEIRQVLSAMRRGENLRFEDYMVFDPLIYQGMAEMHDRHRDMRLDVDNMSYEELLALGERIGDVSTGLSEEVILKVMKQHKHTSSAAGSHQDMEPCCVCQEEYAEGDDLGTLGCGHEFHTACVKQWLMLKNLCPICKTVALST

SEQ ID NO: 124 - DNA, 2148 bp, Glycine max
atgcaaggacaaaggaggactattggatcatttccagcaattgtaagtatgatgcaagggcccagttctagtggcactgatatgagtcatcagtcttccttgaatcatgtgcaaaatgcagtagatttccggttgtcagattacaggggatcttctggtgagactgcatgtttacgtggcactggtcataatgttcagagcttcaatggctggagtactggtgaatctagttctagactgaatctgatcaaccaagtcaatgatgaaggtctaaaatcagaacatgggctgtcttcttcgtataatgctgctactgaggatggtctcaggtctgaggaaagacaattcgaaccaaataatgtaatttttcctgttagttcaaatactaatctacatggcaatcaatctagagtccacccttcctttttgcaaggttccagttctactcgtattagccagaatattagtctagatatggggcatgttactaatgctgctgatcgtgggaagggcaaagaagctggtagtagtgttaatgccaacaatactagtggaatagatagagaaaagacattgtttggcagtgcttcttgtaatcacataggagcttcatctgaaagctctggatacatggctcagggtgatagtgctaattcaagttcttctttagttaattggggtccttcctgcaagagaaaggctcttgaaggtagttctaggcaactgtgctctggaggaagctcaagcactcttgtacaatctggtaatggttgctggcctattgaccctgttgatcttaatgcttctagcagcttaagtgattctacacctatagaagatattcctgttactagtcctccattatttcagaatgcaagaaatgaagtgagacaagaagcttccaatgcatttcctttgataagtattgcagaaaatgtggaaaggcctctaagaaactttgatagaagaatgggccatctacagcatcaggaatctgtacctctcaatttaccatcaacagggagtgctaggcatcataatcattcttctctgcatcaaatacctggctctcactcaatcaatgattcattggaattgaggttaacagctggagtgtcatctgctaattctggtgcttctctgaaccaatcacctgccttgcgcatccattcttttccttggaacagaactgccaatcgtagaggggccaggtcttcaacttcttataactctggagaaagagctgtctgggaagattttaatttgagaatgtttccaagagatagtactgaacaccccatgaatgtgcctgcgtcttcaggacatgaacctactggttggcatacaccatctggtaatgtgaataattctggaggtgtacctcctccatcctggattggatctagttcaaatgttcactctccccctaaccctagctggatttttaatcatgaagtcccagcagaaaatatgcagagtgtgtcagagttcagtccctggtccctttttccatcaattagctctgcatctggtgttcataatggccattcagccccatcttcttctggtcctccttcatttacccagggttctagcagcaaccaaccatatgcaagaacagcattgttgatggaaagaaggggtggtgatgttctctctggtccccattcactgcgagcattaacctttgacaatgaggggagacgtcgactaatatctgagattcgccaagtcttgatggcaatgcggaggggtgagaacttacgagctgaggattatatgctctttgaccctttcctatatcatggcatggctgaaatgcatgacaggcacagagaaatgcgccttgatgtcgacaacatgtcttatgaggagttgttggcattggaggagcgtataggagacgtgagcactggattgagtgaggacatcattattaagttgatgaaacaacgaatttacgtgtctgtcatgacagactcttctattgatttggaaccttgctgtatctgtcaggatgaatttgctgatggagagaatgttggatcactggattgtgggcatgagttccatagtggctgcatcaagcagtggctaatgcagaagaacctctgccctatttgcaaaacaacagccttagctacttga

SEQ ID NO: 125 - protein, 715 aa, Glycine max
MQGQRRTIGSFPAIVSMMQGPSSSGTDMSHQSSLNHVQNAVDFRLSDYRGSSGETACLRGTGHNVQSFNGWSTGESSSRLNLINQVNDEGLKSEHGLSSSYNAATEDGLRSEERQFEPNNVIFPVSSNTNLHGNQSRVHPSFLQGSSSTRISQNISLDMGHVTNAADRGKGKEAGSSVNANNTSGIDREKTLFGSASCNHIGASSESSGYMAQGDSANSSSSLVNWGPSCKRKALEGSSRQLCSGGSSSTLVQSGNGCWPIDPVDLNASSSLSDSTPIEDIPVTSPPLFQNARNEVRQEASNAFPLISIAENVERPLRNFDRRMGHLQHQESVPLNLPSTGSARHHNHSSLHQIPGSHSINDSLELRLTAGVSSANSGASLNQSPALRIHSFPWNRTANRRGARSSTSYNSGERAVWEDFNLRMFPRDSTEHPMNVPASSGHEPTGWHTPSGNVNNSGGVPPPSWIGSSSNVHSPPNPSWIFNHEVPAENMQSVSEFSPWSLFPSISSASGVHNGHSAPSSSGPPSFTQGSSSNQPYARTALLMERRGGDVLSGPHSLRALTFDNEGRRRLISEIRQVLMAMRRGENLRAEDYMLFDPFLYHGMAEMHDRHREMRLDVDNMSYEELLALEERIGDVSTGLSEDIIIKLMKQRIYVSVMTDSSIDLEPCCICQDEFADGENVGSLDCGHEFHSGCIKQWLMQKNLCPICKTTALAT

SEQ ID NO: 126 - DNA, 1656 bp, Oryza sativa
atggctgctcgccggacttccaaaatattcgagcaagacagcgagttgaggaaagcgcttctcaacagcgttgatcatttctacaacaaggtaatcaagccactactggagtctgatggcggcggtggcggcggcggcggcggcggcggcagcggcggcggtggtggcggcggcggcggcggtggtggcggcggcggcagcggcggcggctgtggcggcggcggcggcggtggcggcggcagcagcggtggcggcggcagcgaggtcctggagcgccgcgttaccgaagtgctccatgtgtacggccttcccggcaagctcccggagctccgccttcccggcgtgctcccgaagccggagccccgccttcccggcgagcccaaacctccgtcgtggatggttttcgtggaagctcccttgccgcccccaagcttgagctacgaagatggcgggaacggcgactccaaccacaccatcaccatggtcgtcgccgacgtccacgccgagccaccgctttcggggtggtggcttgcacgaatactattgcgctggcgccggaagatcgaggaacttccacgccatgtgatctacgtgatcggcgctgcggcgatcgtgggcacaggctacataatctacttgctcgtcaagcggcgtcgtcgacctcgagatgctcgtccgcctctacccgggaatggaggccaacctccacctggaggtgaccatcctcaagcccctaagctcaagcaccttccagatgatagagcggcttcgggtggcgatgatgcagagtacgacgaggatcagggccctggcgacggcgacgagacaggtggtgagggaagcgctgcctacggcctccacgacatcgccgcgtttgctgtcgccttcagcaactcgcctacagggccaaccctggcagttgagaataaccctgccttcctcgctctccaacagattaaagttgccagggagatctgtaacaacaaggctgtgcgcttgttacagttgctgaatcctgagaagtcccacttcagtatcccgtggttcgagaggctgacaatctttgatgtctgcccccgccccaacctcgtcgagagcacttctggaagccgtgatctccagatggtgagacctggtcttggtgtccttacaagaccattaccaactaagtacagatccctgggggataatttctgtgagagagttttgacttcacttatgcatgaaacactcaaagcggtggttgcacagtacaatgccagtcagcttatcatacccagagaaagctttagaaaagagttcactcatgcaattgaaaccaagcaggtcgatgaacaagaagctcagcgtgcaaagttcattgttgagaaggctgaacaacataagaggaaggcagtcattacagaacaggcctgcttgaacagggtgaagctaagagtgcattggtcgagcctgcaactagaagaagaagagctcgggatctttgagaaggtccaggaagtagaagtatcaaaagaatttggatgtagtagttggctattgcaggttacatatctgactagatgcagtcacggttctccagtgatcctggatgtaggtttcaccaccaccgaggaaaacaatacaagccgagatgaatttagtctactgaagtaa

SEQ ID NO: 127 - protein, 551 aa, Oryza sativa
MAARRTSKIFEQDSELRKALLNSVDHFYNKVIKPLLESDGGGGGGGGGGGSGGGGGGGGGGGGGGGSGGGCGGGGGGGGGSSGGGGSEVLERRVTEVLHVYGLPGKLPELRLPGVLPKPEPRLPGEPKPPSWMVFVEAPLPPPSLSYEDGGNGDSNHTITMVVADVHAEPPLSGWWLARILLRWRRKIEELPRHVIYVIGAAAIVGTGYIIYLLVKRRRRPRDARPPLPGNGGQPPPGGDHPQAPKLKHLPDDRAASGGDDAEYDEDQGPGDGDETGGEGSAAYGLHDIAAFAVAFSNSPTGPTLAVENNPAFLALQQIKVAREICNNKAVRLLQLLNPEKSHFSIPWFERLTIFDVCPRPNLVESTSGSRDLQMVRPGLGVLTRPLPTKYRSLGDNFCERVLTSLMHETLKAVVAQYNASQLIIPRESFRKEFTHAIETKQVDEQEAQRAKFIVEKAEQHKRKAVITEQACLNRVKLRVHWSSLQLEEEELGIFEKVQEVEVSKEFGCSSWLLQVTYLTRCSHGSPVILDVGFTTTEENNTSRDEFSLLK